SEQUENCE LISTING (49 sequences)

SEQ ID NO: 1 - protein, 131 aa, Canis lupus
MGLTSQLIPTLVCLLALTSTFVHGHNFNITIKEIIKMLNILTARNDSCMELTVDVFTAPKNTSDKEIFCRAATVLRQIYTHNCSNRYLRGLYRNLSSMANKTCSMNEIKKSTLKDFLERLKVIMQKKYYRH

SEQ ID NO: 2 - protein, 132 aa, Felis catus
MDLTSQLIPALVCLLAFTSTFVHGQNFNNTLKEIIKTLNILTARNDSCMELTMDVLAAPKNTSDKEIFCRATTVLRQIYTHHNCSTKFLKGLDRNLSSMANRTCSVNEVKKCTLKDFLERLKAIMQKKYSKH

SEQ ID NO: 3 - protein, 137 aa, Equus caballus
MGLTYQLIPALVCLLACTSNFIQGCKYDITLQEIIKTLNNLTDGKGKNSCMELTVADAFAGPKNTDGKEICRAAKVLQQLYKRHDRSLIKECLSGLDRNLKGMANGTCCTVNEAKKSTLKDFLERLKTIMKEKYSKC

SEQ ID NO: 4 - protein, 131 aa, Canis lupus
MALWLTVVIALTCLGGLASPSPVTPSPTLKELIEELVNITQNQASLCNGSMVWSVNLTAGMYCAALESLINVSDCSAIQRTQRMLKALCSQKPAAGQISSERSRDTKIEVIQLVKNLLTYVRGVYRHGNFR

SEQ ID NO: 5 - protein, 183 aa, Felis catus
MWFLDSTRQSGDQGGRRHTWPIKATARGQGHKPLSLGQPTCPLLAPPVLALGSMALWLTVVIALTCLGGLASPGPHSRRELKELIEELVNITQNQVSLCNGSMVWSVNLTTGMYCAALESLINVSDCTAIQRTQRMLKALCTQKPSAGQTASERSRDTKIEVIQLVKNLLNHLRRNFRHGNFK

SEQ ID NO: 6 - protein, 133 aa, Equus caballus
MALWLTAVIALACLGGLASPAPLPSSMALKELIKELVNITQNQAPLCNGSMVWSVNLTADTYCRALESLSNVSTCSAIQNTRKMLTKLCPHQLSAGQVSSERARDTKIEVIVLVKDLLKNLRKIFHGGKHVDA

SEQ ID NO: 7 - protein, 823 aa, Canis lupus
MGRLCSGLTFPVSCLVLVWVASSGSVKVLHEPSCFSDYISTSVCQWKMDHPTNCSAELRLSYQLDFMGSENHTCVPENREDSVCVCSMPIDDAVEADVYQLDLWAGQQLLWSGSFQPSKHVKPRTPGNLTVHPNISHTWLLMWTNPYPTENHLHSELTYMVNVSNDNDPEDFKVYNVTYMGPTLRLAASTLKSGASYSARVRAWAQTYNSTWSDWSPSTTWLNYYEPWEQHLPLGVSISCLVILAICLSCYFSIIKIKKGWWDQIPNPAHSPLVAIVIQDSQVSLWGKRSRGQEPAKCPHWKTCLTKLLPCLLEHGLGREEESPKTAKNGPLQGPGKPAWCPVEVSKTILWPESISVVQCVELSEAPVDNEEEEEVEEDKRSLCPSLEGSGGSFQEGREGIVARLTESLFLDLLGGENGGFCPQGLEESCLPPPSGSVGAQMPWAQFPRAGPRAAPEGPEQPRRPESALQASPTQSAGSSAFPEPPPVVTDNPAYRSFGSFLGQSSDPGDGDSDPELADRPGEADPGIPSAPQPPEPPAALQPEPESWEQILRQSVLQHRAAPAPGPGPGSGYREFTCAVKQGSAPDAGGPGFGPSGEAGYKAFCSLLPGGATCPGTSGGEAGSGEGGYKPFQSLTPGCPGAPTPVPVPLFTFGLDTEPPGSPQDSLGAGSSPEHLGVEPAGKEEDSRKTLLAPEQATDPLRDDLASSIVYSALTCHLCGHLKQWHDQEERGKAHIVPSPCCGCCCGDRSSLLLSPLRAPNVLPGGVLLEASLSPASLVPSGVSKEGKSSPFSQPASSSAQSSSQTPKKLAVLSTEPTCMSAS

SEQ ID NO: 8 - protein, 881 aa, Felis catus
MGRLCSGLTFPVSCLILMWAAGSGSVKVLRAPTCFSDYFSTSVCQWNMDAPTNCSAELRLSYQLNFMGSENRTCVPENGEGAACACSMLMDDFVEADVYQLHLWAGTQLLWSGSFKPSSHVKPRAPGNLTVHPNVSHTWLLRWSNPYPPENHLHAELTYMVNISSEDDPTDVSVCASGFLCHLLGLRRVETGAPGARLPPWLCAPRPRRVPGSQCAVISCCRWVLIALTSRGGRWRLTPGLRSQTRYVSVAEGLFGATPRVLCPGTQAGLASAAREQMSPDPSAFHSIDYEPWEQHLPLGVSISCLVILAVCLSCYLSVIKIKKEWWDQIPNPAHSHLVAIVIQDPQVSLWGKRSRGQEPAKCPHWKTCLRKLLPCLLEHGMERKEDPSKIARNGPSQCSGKSAWCPVEVSKTILWPESISVVRCVELLEAPVESEEEEEEEEDKGSFCPSPVNLEDSFQEGREGIAARLTESLFMDLLGVEKGGFGPQGSLESWFPPPSGSAGAQMPWAEFPGPGPQEASPQGKEQPFDPRSDPLATLPQSPASPTFPETPPVVTDNPAYRSFGTFQGRSSGPGECDSGPELAGRLGEADPGIPAAPQPSEPPSALQPEAETWEQILRQRVLQHRGAPAPAPGSGYREFVCAVRQGSTQDSGVGDFGPSEEAGYKAFSSLLTSGAVCPESGGEAGSGDGGYKPFQSLTPGCPGAPAPVPVPLFTFGLDAEPPHCPQDSPLPGSSPEPAGKAQDSHKTPPAPEQAADPLRDDLASGIVYSALTCHLCGHLKQCHGQEEGGEAHPVASPCCGCCCGDRSSPLVSPLRAPDPLPGGVPLEASLSPASPAPLAVSEEGPPSLCFQPALSHAHSSSQTPKKVAMLSPEPTCTMAS

SEQ ID NO: 9 - protein, 809 aa, Equus caballus
MGCLCPGLTLPVSCLILVWAAGSGSVKVLHLTACFSDYISASTCEWKMDRPTNCSAQLRLSYQLNDEFSDNLTCIPENREDEVCVCRMLMDNIVSEDVYELDLWAGNQLLWNSSFKPSRHVKPRAPQNLTVHAISHTWLLTWSNPYPLKNHLWSELTYLVNISKEDDPTDFKIYNVTYMDPTLRVTASTLKSRATYSARVKARAQNYNSTWSEWSPSTTWHNYYEQPLEQRLPLGVSISCVVILAICLSCYFSIIKIKKEWWDQIPNPAHSPLVAIVLQDSQVSLWGKQSRGQEPAKCPRWKTCLTKLLPCLLEHGLQKEEDSSKTVRNGPFQSPGKSAWHTVEVNHTILRPEIISVVPCVELCEAQVESEEEEVEEDRGSFCPSPESSGSGFQEGREGVAARLTESLFLGLLGAENGALGESCLLPPLGSAHMPWARISSAGPQEAASQGEEQPLNPESNPLATLTQSPGSLAFTEAPAVVADNPAYRSFSNSLSQPRGPGELDSDPQLAEHLGQVDPSIPSAPQPSEPPTALQPEPETWEQMLRQSVLQQGAAPAPASAPTGGYREFAQAVKQGGGAAGSGPSGEAGYKAFSSLLAGSAVCPGQSGVEASSGEGGYRPYESPDPGAPAPVPVPLFTFGLDVEPPHSPQNSLLPGGSPELPGPEPTVKGEDPRKPLLSAQQATDSLRDDLGSGIVYSALTCHLCGHLKQCHGQEEHGEAHTVASPCCGCCCGDRSSPPVSPVRALDPPPGGVPLEAGLSLASLGSLGLSEERKPSLFFQPAPGNAQSSSQTPLTVAMLSTGPTCTSAS

SEQ ID NO: 10 - protein, 427 aa, Canis lupus
MERPARLCGLWALLLCAAGGRGGGVAAPTETQPPVTNLSVSVENLCTVIWTWDPPEGASPNCTLRYFSHFDNKQDKKIAPETHRSKEVPLNERICLQVGSQCSTNESDNPSILVEKCTPPPEGDPESAVTELQCVWHNLSYMKCTWLPGRNTSPDTNYTLYYWHSSLGKILQCEDIYREGQHIGCSFALTNLKDSSFEQHSVQIVVKDNAGKIRPSFNIVPLTSHVKPDPPHIKRLFFQNGNLYVQWKNPQNFYSRCLSYQVEVNNSQTETNDIFYVEEAKCQNSEFEGNLEGTICFMVPGVLPDTLNTVRIRVRTNKLCYEDDKLWSNWSQAMSIGENTDPTFYITMLLATPVIVAGAIIVLLLYLKRLKIIIFPPIPDPGKIFKEMFGDQNDDTLHWRKYDIYEKQTKEETDSVVLIENLKKASQ

SEQ ID NO: 11 - protein, 479 aa, Felis catus
MMTKCSSDRNVFKRKWFLFPASQYTFRPIHQARPCEVPAVHLEPSPPWEVGLGLLNLESEFRKLGLRGRRLAAAPPDSRAEAASQTQPPVTNLSVSVENLCTVIWTWDPPEGASPNCTLRYFSHFDNKQDKKIAPETHRSKEVPLNERICLQVGSQCSTNESDNPSILVEKCTPPPEGDPESAVTELQCVWHNLSYMKCTWLPGRNTSPDTNYTLYYWHSSLGKILQCENIYREGQHIGCSFALTNLKDSSFEQHSVQIVVKDNAGKIRPSFNIVPLTSHVKPDPPHIKRLFFQNGNLYVQWKNPQNFYSRCLSYQVEVNNSQTETHDIFYVEEAKCQNSEFEGNLEGTICFMVPGILPDTLNTVRIRVRTNKLCYEDDRLWSNWSQAMSIGENTDPTFYITMLLATPVIVAGAIIVLLLYLKRLKIIIFPPIPDPGKIFKEMFGDQNDDSLHWKKYDIYEKQTKEETDSVVLIENASQ

SEQ ID NO: 12 - protein, 407 aa, Equus caballus
MYFLCLIWTESQPPVTNLSVSVENLCTVIWTWNPPEGVSPNCSLWYFSHFGNKQDKKIAPETHRSKEVPLNERICLQVGSQCSTNESDNPSILVEKCISPPEGDPESAVTELQCVWHNLSYMKCTWLPGKNASPDTNYTLYYWHSSLGKILQCEDIYREGQHIGCSFALTEVKDSIFEQHSVQIMVKDNAGKIRPFFNIVPLTSHVKPDPPHIKKLFFQNGDLYVQWKNPQNFYSRCLSYQVEVNNSQTETRDIFSVEEAKCQNPEFEGDLEGTICFMVPGVLPDTVNTVRIRVKTNKLCYEDDKLWSNWSQAMSIGKKADPTFYIAMLLIIPVIVAGAIIVLLLYLKRLKIIMFPPIPDPGKIFKEMFGDQNDDTLHWKKYDIYEKQTKEETDSVVLIENLKRASQ

SEQ ID NO: 13 - protein, 761 aa, synthetic construct
  REGION 1..761: note = Canis lupus IL13R-IL4R-Fc (without signal sequence)
TETQPPVTNLSVSVENLCTVIWTWDPPEGASPNCTLRYFSHFDNKQDKKIAPETHRSKEVPLNERICLQVGSQCSTNESDNPSILVEKCTPPPEGDPESAVTELQCVWHNLSYMKCTWLPGRNTSPDTNYTLYYWHSSLGKILQCEDIYREGQHIGCSFALTNLKDSSFEQHSVQIVVKDNAGKIRPSFNIVPLTSHVKPDPPHIKRLFFQNGNLYVQWKNPQNFYSRCLSYQVEVNNSQTETNDIFYVEEAKCQNSEFEGNLEGTICFMVPGVLPDTLNTVRIRVRTNKLCYEDDKLWSNWSQAMSIGENTDPTGGGSGSGSVKVLHEPSCFSDYISTSVCQWKMDHPTNCSAELRLSYQLDFMGSENHTCVPENREDSVCVCSMPIDDAVEADVYQLDLWAGQQLLWSGSFQPSKHVKPRTPGNLTVHPNISHTWLLMWTNPYPTENHLHSELTYMVNVSNDNDPEDFKVYNVTYMGPTLRLAASTLKSGASYSARVRAWAQTYNSTWSDWSPSTTWLNYYEPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 14 - protein, 758 aa, synthetic construct
  REGION 1..758: note = Canis lupus IL4R-IL13R-IgGA (without signal sequence)
SGSVKVLHEPSCFSDYISTSVCQWKMDHPTNCSAELRLSYQLDFMGSENHTCVPENREDSVCVCSMPIDDAVEADVYQLDLWAGQQLLWSGSFQPSKHVKPRTPGNLTVHPNISHTWLLMWTNPYPTENHLHSELTYMVNVSNDNDPEDFKVYNVTYMGPTLRLAASTLKSGASYSARVRAWAQTYNSTWSDWSPSTTWLNYYEPGGGSGTETQPPVTNLSVSVENLCTVIWTWDPPEGASPNCTLRYFSHFDNKQDKKIAPETHRSKEVPLNERICLQVGSQCSTNESDNPSILVEKCTPPPEGDPESAVTELQCVWHNLSYMKCTWLPGRNTSPDTNYTLYYWHSSLGKILQCEDIYREGQHIGCSFALTNLKDSSFEQHSVQIVVKDNAGKIRPSFNIVPLTSHVKPDPPHIKRLFFQNGNLYVQWKNPQNFYSRCLSYQVEVNNSQTETNDIFYVEEAKCQNSEFEGNLEGTICFMVPGVLPDTLNTVRIRVRTNKLCYEDDKLWSNWSQAMSIGENTDPTFNECRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 15 - protein, 762 aa, synthetic construct
  REGION 1..762: note = Canis lupus IL4R-IL13R-IgGB (without signal sequence)
SGSVKVLHEPSCFSDYISTSVCQWKMDHPTNCSAELRLSYQLDFMGSENHTCVPENREDSVCVCSMPIDDAVEADVYQLDLWAGQQLLWSGSFQPSKHVKPRTPGNLTVHPNISHTWLLMWTNPYPTENHLHSELTYMVNVSNDNDPEDFKVYNVTYMGPTLRLAASTLKSGASYSARVRAWAQTYNSTWSDWSPSTTWLNYYEPGGGSGTETQPPVTNLSVSVENLCTVIWTWDPPEGASPNCTLRYFSHFDNKQDKKIAPETHRSKEVPLNERICLQVGSQCSTNESDNPSILVEKCTPPPEGDPESAVTELQCVWHNLSYMKCTWLPGRNTSPDTNYTLYYWHSSLGKILQCEDIYREGQHIGCSFALTNLKDSSFEQHSVQIVVKDNAGKIRPSFNIVPLTSHVKPDPPHIKRLFFQNGNLYVQWKNPQNFYSRCLSYQVEVNNSQTETNDIFYVEEAKCQNSEFEGNLEGTICFMVPGVLPDTLNTVRIRVRTNKLCYEDDKLWSNWSQAMSIGENTDPTPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 16 - protein, 760 aa, synthetic construct
  REGION 1..760: note = Canis lupus IL4R-IL13R-IgGC (without signal sequence)
SGSVKVLHEPSCFSDYISTSVCQWKMDHPTNCSAELRLSYQLDFMGSENHTCVPENREDSVCVCSMPIDDAVEADVYQLDLWAGQQLLWSGSFQPSKHVKPRTPGNLTVHPNISHTWLLMWTNPYPTENHLHSELTYMVNVSNDNDPEDFKVYNVTYMGPTLRLAASTLKSGASYSARVRAWAQTYNSTWSDWSPSTTWLNYYEPGGGSGTETQPPVTNLSVSVENLCTVIWTWDPPEGASPNCTLRYFSHFDNKQDKKIAPETHRSKEVPLNERICLQVGSQCSTNESDNPSILVEKCTPPPEGDPESAVTELQCVWHNLSYMKCTWLPGRNTSPDTNYTLYYWHSSLGKILQCEDIYREGQHIGCSFALTNLKDSSFEQHSVQIVVKDNAGKIRPSFNIVPLTSHVKPDPPHIKRLFFQNGNLYVQWKNPQNFYSRCLSYQVEVNNSQTETNDIFYVEEAKCQNSEFEGNLEGTICFMVPGVLPDTLNTVRIRVRTNKLCYEDDKLWSNWSQAMSIGENTDPTAKECECKCNCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 17 - protein, 758 aa, synthetic construct
  REGION 1..758: note = Canis lupus IL4R-IL13R-IgGD (without signal sequence)
SGSVKVLHEPSCFSDYISTSVCQWKMDHPTNCSAELRLSYQLDFMGSENHTCVPENREDSVCVCSMPIDDAVEADVYQLDLWAGQQLLWSGSFQPSKHVKPRTPGNLTVHPNISHTWLLMWTNPYPTENHLHSELTYMVNVSNDNDPEDFKVYNVTYMGPTLRLAASTLKSGASYSARVRAWAQTYNSTWSDWSPSTTWLNYYEPGGGSGTETQPPVTNLSVSVENLCTVIWTWDPPEGASPNCTLRYFSHFDNKQDKKIAPETHRSKEVPLNERICLQVGSQCSTNESDNPSILVEKCTPPPEGDPESAVTELQCVWHNLSYMKCTWLPGRNTSPDTNYTLYYWHSSLGKILQCEDIYREGQHIGCSFALTNLKDSSFEQHSVQIVVKDNAGKIRPSFNIVPLTSHVKPDPPHIKRLFFQNGNLYVQWKNPQNFYSRCLSYQVEVNNSQTETNDIFYVEEAKCQNSEFEGNLEGTICFMVPGVLPDTLNTVRIRVRTNKLCYEDDKLWSNWSQAMSIGENTDPTPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 18 - protein, 590 aa, synthetic construct
  REGION 1..590: note = Felis catus IL4R-IL13R (without signal sequence)
SGSVKVLRAPTCFSDYFSTSVCQWNMDAPTNCSAELRLSYQLNFMGSENRTCVPENGEGAACACSMLMDDFVEADVYQLHLWAGTQLLWSGSFKPSSHVKPRAPGNLTVHPNVSHTWLLRWSNPYPPENHLHAELTYMVNISSEDDPTDVSVCASGFLCHLLGLRRVETGAPGARLPPWLCAPRPRRVPGSQCAVISCCRWVLIALTSRGGRWRLTPGLRSQTRYVSVAEGLFGATPRVLCPGTQAGLASAAREQMSPDPSAFHSIDYEPGGGSGSQTQPPVTNLSVSVENLCTVIWTWDPPEGASPNCTLRYFSHFDNKQDKKIAPETHRSKEVPLNERICLQVGSQCSTNESDNPSILVEKCTPPPEGDPESAVTELQCVWHNLSYMKCTWLPGRNTSPDTNYTLYYWHSSLGKILQCENIYREGQHIGCSFALTNLKDSSFEQHSVQIVVKDNAGKIRPSFNIVPLTSHVKPDPPHIKRLFFQNGNLYVQWKNPQNFYSRCLSYQVEVNNSQTETHDIFYVEEAKCQNSEFEGNLEGTICFMVPGILPDTLNTVRIRVRTNKLCYEDDRLWSNWSQAMSIGENTDPT

SEQ ID NO: 19 - protein, 525 aa, synthetic construct
  REGION 1..525: note = Equus caballus IL4R-IL13R (without signal sequence)
SGSVKVLHLTACFSDYISASTCEWKMDRPTNCSAQLRLSYQLNDEFSDNLTCIPENREDEVCVCRMLMDNIVSEDVYELDLWAGNQLLWNSSFKPSRHVKPRAPQNLTVHAISHTWLLTWSNPYPLKNHLWSELTYLVNISKEDDPTDFKIYNVTYMDPTLRVTASTLKSRATYSARVKARAQNYNSTWSEWSPSTTWHNYYEQPGGGSGTESQPPVTNLSVSVENLCTVIWTWNPPEGVSPNCSLWYFSHFGNKQDKKIAPETHRSKEVPLNERICLQVGSQCSTNESDNPSILVEKCISPPEGDPESAVTELQCVWHNLSYMKCTWLPGKNASPDTNYTLYYWHSSLGKILQCEDIYREGQHIGCSFALTEVKDSIFEQHSVQIMVKDNAGKIRPFFNIVPLTSHVKPDPPHIKKLFFQNGDLYVQWKNPQNFYSRCLSYQVEVNNSQTETRDIFSVEEAKCQNPEFEGDLEGTICFMVPGVLPDTVNTVRIRVKTNKLCYEDDKLWSNWSQAMSIGKKADPT

SEQ ID NO: 20 - protein, 780 aa, synthetic construct
  REGION 1..780: note = Canis lupus IL13R-IL4R-IgGB (with signal sequence)
MAVLGLLFCLVTFPSCVLSTETQPPVTNLSVSVENLCTVIWTWDPPEGASPNCTLRYFSHFDNKQDKKIAPETHRSKEVPLNERICLQVGSQCSTNESDNPSILVEKCTPPPEGDPESAVTELQCVWHNLSYMKCTWLPGRNTSPDTNYTLYYWHSSLGKILQCEDIYREGQHIGCSFALTNLKDSSFEQHSVQIVVKDNAGKIRPSFNIVPLTSHVKPDPPHIKRLFFQNGNLYVQWKNPQNFYSRCLSYQVEVNNSQTETNDIFYVEEAKCQNSEFEGNLEGTICFMVPGVLPDTLNTVRIRVRTNKLCYEDDKLWSNWSQAMSIGENTDPTGGGSGSGSVKVLHEPSCFSDYISTSVCQWKMDHPTNCSAELRLSYQLDFMGSENHTCVPENREDSVCVCSMPIDDAVEADVYQLDLWAGQQLLWSGSFQPSKHVKPRTPGNLTVHPNISHTWLLMWTNPYPTENHLHSELTYMVNVSNDNDPEDFKVYNVTYMGPTLRLAASTLKSGASYSARVRAWAQTYNSTWSDWSPSTTWLNYYEPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 21 - protein, 781 aa, synthetic construct
  REGION 1..781: note = Canis lupus IL4R-IL13R-IgGB (with signal sequence),
MAVLGLLFCLVTFPSCVLSSGSVKVLHEPSCFSDYISTSVCQWKMDHPTNCSAELRLSYQLDFMGSENHTCVPENREDSVCVCSMPIDDAVEADVYQLDLWAGQQLLWSGSFQPSKHVKPRTPGNLTVHPNISHTWLLMWTNPYPTENHLHSELTYMVNVSNDNDPEDFKVYNVTYMGPTLRLAASTLKSGASYSARVRAWAQTYNSTWSDWSPSTTWLNYYEPGGGSGTETQPPVTNLSVSVENLCTVIWTWDPPEGASPNCTLRYFSHFDNKQDKKIAPETHRSKEVPLNERICLQVGSQCSTNESDNPSILVEKCTPPPEGDPESAVTELQCVWHNLSYMKCTWLPGRNTSPDTNYTLYYWHSSLGKILQCEDIYREGQHIGCSFALTNLKDSSFEQHSVQIVVKDNAGKIRPSFNIVPLTSHVKPDPPHIKRLFFQNGNLYVQWKNPQNFYSRCLSYQVEVNNSQTETNDIFYVEEAKCQNSEFEGNLEGTICFMVPGVLPDTLNTVRIRVRTNKLCYEDDKLWSNWSQAMSIGENTDPTPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 22 - protein, 315 aa, synthetic construct
  REGION 1..315: note = Canis lupus IL13R extracellular domain (without signal sequence)
TETQPPVTNLSVSVENLCTVIWTWDPPEGASPNCTLRYFSHFDNKQDKKIAPETHRSKEVPLNERICLQVGSQCSTNESDNPSILVEKCTPPPEGDPESAVTELQCVWHNLSYMKCTWLPGRNTSPDTNYTLYYWHSSLGKILQCEDIYREGQHIGCSFALTNLKDSSFEQHSVQIVVKDNAGKIRPSFNIVPLTSHVKPDPPHIKRLFFQNGNLYVQWKNPQNFYSRCLSYQVEVNNSQTETNDIFYVEEAKCQNSEFEGNLEGTICFMVPGVLPDTLNTVRIRVRTNKLCYEDDKLWSNWSQAMSIGENTDPT

SEQ ID NO: 23 - protein, 207 aa, synthetic construct
  REGION 1..207: note = Canis lupus IL4R extracellular domain (without signal sequence)
SGSGSVKVLHEPSCFSDYISTSVCQWKMDHPTNCSAELRLSYQLDFMGSENHTCVPENREDSVCVCSMPIDDAVEADVYQLDLWAGQQLLWSGSFQPSKHVKPRTPGNLTVHPNISHTWLLMWTNPYPTENHLHSELTYMVNVSNDNDPEDFKVYNVTYMGPTLRLAASTLKSGASYSARVRAWAQTYNSTWSDWSPSTTWLNYYEP

SEQ ID NO: 24 - protein, 315 aa, synthetic construct
  REGION 1..315: note = Felis catus IL13R extracellular domain (without signal sequence)
SQTQPPVTNLSVSVENLCTVIWTWDPPEGASPNCTLRYFSHFDNKQDKKIAPETHRSKEVPLNERICLQVGSQCSTNESDNPSILVEKCTPPPEGDPESAVTELQCVWHNLSYMKCTWLPGRNTSPDTNYTLYYWHSSLGKILQCENIYREGQHIGCSFALTNLKDSSFEQHSVQIVVKDNAGKIRPSFNIVPLTSHVKPDPPHIKRLFFQNGNLYVQWKNPQNFYSRCLSYQVEVNNSQTETHDIFYVEEAKCQNSEFEGNLEGTICFMVPGILPDTLNTVRIRVRTNKLCYEDDRLWSNWSQAMSIGENTDPT

SEQ ID NO: 25 - protein, 270 aa, synthetic construct
  REGION 1..270: note = Felis catus IL4R extracellular domain (without signal sequence)
SGSVKVLRAPTCFSDYFSTSVCQWNMDAPTNCSAELRLSYQLNFMGSENRTCVPENGEGAACACSMLMDDFVEADVYQLHLWAGTQLLWSGSFKPSSHVKPRAPGNLTVHPNVSHTWLLRWSNPYPPENHLHAELTYMVNISSEDDPTDVSVCASGFLCHLLGLRRVETGAPGARLPPWLCAPRPRRVPGSQCAVISCCRWVLIALTSRGGRWRLTPGLRSQTRYVSVAEGLFGATPRVLCPGTQAGLASAAREQMSPDPSAFHSIDYEP

SEQ ID NO: 26 - protein, 315 aa, synthetic construct
  REGION 1..315: note = Equus caballus IL13R extracellular domain (without signal sequence)
TESQPPVTNLSVSVENLCTVIWTWNPPEGVSPNCSLWYFSHFGNKQDKKIAPETHRSKEVPLNERICLQVGSQCSTNESDNPSILVEKCISPPEGDPESAVTELQCVWHNLSYMKCTWLPGKNASPDTNYTLYYWHSSLGKILQCEDIYREGQHIGCSFALTEVKDSIFEQHSVQIMVKDNAGKIRPFFNIVPLTSHVKPDPPHIKKLFFQNGDLYVQWKNPQNFYSRCLSYQVEVNNSQTETRDIFSVEEAKCQNPEFEGDLEGTICFMVPGVLPDTVNTVRIRVKTNKLCYEDDKLWSNWSQAMSIGKKADPT

SEQ ID NO: 27 - protein, 205 aa, synthetic construct
  REGION 1..205: note = Equus caballus IL4R extracellular domain (without signal sequence)
SGSVKVLHLTACFSDYISASTCEWKMDRPTNCSAQLRLSYQLNDEFSDNLTCIPENREDEVCVCRMLMDNIVSEDVYELDLWAGNQLLWNSSFKPSRHVKPRAPQNLTVHAISHTWLLTWSNPYPLKNHLWSELTYLVNISKEDDPTDFKIYNVTYMDPTLRVTASTLKSRATYSARVKARAQNYNSTWSEWSPSTTWHNYYEQP

SEQ ID NO: 28 - protein, 829 aa, synthetic construct
  REGION 1..829: note = Feline IL13R-IL4R-IgG2 (without signal sequence)
SQTQPPVTNLSVSVENLCTVIWTWDPPEGASPNCTLRYFSHFDNKQDKKIAPETHRSKEVPLNERICLQVGSQCSTNESDNPSILVEKCTPPPEGDPESAVTELQCVWHNLSYMKCTWLPGRNTSPDTNYTLYYWHSSLGKILQCENIYREGQHIGCSFALTNLKDSSFEQHSVQIVVKDNAGKIRPSFNIVPLTSHVKPDPPHIKRLFFQNGNLYVQWKNPQNFYSRCLSYQVEVNNSQTETHDIFYVEEAKCQNSEFEGNLEGTICFMVPGILPDTLNTVRIRVRTNKLCYEDDRLWSNWSQAMSIGENTDPTGGGSGSSGSVKVLRAPTCFSDYFSTSVCQWNMDAPTNCSAELRLSYQLNFMGSENRTCVPENGEGAACACSMLMDDFVEADVYQLHLWAGTQLLWSGSFKPSSHVKPRAPGNLTVHPNVSHTWLLRWSNPYPPENHLHAELTYMVNISSEDDPTDVSVCASGFLCHLLGLRRVETGAPGARLPPWLCAPRPRRVPGSQCAVISCCRWVLIALTSRGGRWRLTPGLRSQTRYVSVAEGLFGATPRVLCPGTQAGLASAAREQMSPDPSAFHSIDYEPSPKTASTIESKTGECPKCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 29 - protein, 829 aa, synthetic construct
  REGION 1..829: note = Feline IL4R-IL13R-IgG2 (without signal sequence)
SGSVKVLRAPTCFSDYFSTSVCQWNMDAPTNCSAELRLSYQLNFMGSENRTCVPENGEGAACACSMLMDDFVEADVYQLHLWAGTQLLWSGSFKPSSHVKPRAPGNLTVHPNVSHTWLLRWSNPYPPENHLHAELTYMVNISSEDDPTDVSVCASGFLCHLLGLRRVETGAPGARLPPWLCAPRPRRVPGSQCAVISCCRWVLIALTSRGGRWRLTPGLRSQTRYVSVAEGLFGATPRVLCPGTQAGLASAAREQMSPDPSAFHSIDYEPGGGSGSSQTQPPVTNLSVSVENLCTVIWTWDPPEGASPNCTLRYFSHFDNKQDKKIAPETHRSKEVPLNERICLQVGSQCSTNESDNPSILVEKCTPPPEGDPESAVTELQCVWHNLSYMKCTWLPGRNTSPDTNYTLYYWHSSLGKILQCENIYREGQHIGCSFALTNLKDSSFEQHSVQIVVKDNAGKIRPSFNIVPLTSHVKPDPPHIKRLFFQNGNLYVQWKNPQNFYSRCLSYQVEVNNSQTETHDIFYVEEAKCQNSEFEGNLEGTICFMVPGILPDTLNTVRIRVRTNKLCYEDDRLWSNWSQAMSIGENTDPTSPKTASTIESKTGECPKCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 30 - protein, 754 aa, synthetic construct
  REGION 1..754: note = Equine IL13R-IL4R-IgG2 (without signal sequence)
TESQPPVTNLSVSVENLCTVIWTWNPPEGVSPNCSLWYFSHFGNKQDKKIAPETHRSKEVPLNERICLQVGSQCSTNESDNPSILVEKCISPPEGDPESAVTELQCVWHNLSYMKCTWLPGKNASPDTNYTLYYWHSSLGKILQCEDIYREGQHIGCSFALTEVKDSIFEQHSVQIMVKDNAGKIRPFFNIVPLTSHVKPDPPHIKKLFFQNGDLYVQWKNPQNFYSRCLSYQVEVNNSQTETRDIFSVEEAKCQNPEFEGDLEGTICFMVPGVLPDTVNTVRIRVKTNKLCYEDDKLWSNWSQAMSIGKKADPTGGGSGSSGSVKVLHLTACFSDYISASTCEWKMDRPTNCSAQLRLSYQLNDEFSDNLTCIPENREDEVCVCRMLMDNIVSEDVYELDLWAGNQLLWNSSFKPSRHVKPRAPQNLTVHAISHTWLLTWSNPYPLKNHLWSELTYLVNISKEDDPTDFKIYNVTYMDPTLRVTASTLKSRATYSARVKARAQNYNSTWSEWSPSTTWHNYYEQPDMSKCPKCPAPELLGGPSVFIFPPNPKDTLMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHYTKTDISESLGK

SEQ ID NO: 31 - protein, 754 aa, synthetic construct
  REGION 1..754: note = Equine IL4R-IL13R-IgG2 (without signal sequence)
SGSVKVLHLTACFSDYISASTCEWKMDRPTNCSAQLRLSYQLNDEFSDNLTCIPENREDEVCVCRMLMDNIVSEDVYELDLWAGNQLLWNSSFKPSRHVKPRAPQNLTVHAISHTWLLTWSNPYPLKNHLWSELTYLVNISKEDDPTDFKIYNVTYMDPTLRVTASTLKSRATYSARVKARAQNYNSTWSEWSPSTTWHNYYEQPGGGSGSTESQPPVTNLSVSVENLCTVIWTWNPPEGVSPNCSLWYFSHFGNKQDKKIAPETHRSKEVPLNERICLQVGSQCSTNESDNPSILVEKCISPPEGDPESAVTELQCVWHNLSYMKCTWLPGKNASPDTNYTLYYWHSSLGKILQCEDIYREGQHIGCSFALTEVKDSIFEQHSVQIMVKDNAGKIRPFFNIVPLTSHVKPDPPHIKKLFFQNGDLYVQWKNPQNFYSRCLSYQVEVNNSQTETRDIFSVEEAKCQNPEFEGDLEGTICFMVPGVLPDTVNTVRIRVKTNKLCYEDDKLWSNWSQAMSIGKKADPTDMSKCPKCPAPELLGGPSVFIFPPNPKDTLMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHYTKTDISESLGK

SEQ ID NO: 32 - protein, 305 aa, synthetic construct
  REGION 1..305: note = Canine mini-IL13R ECD
QPPVTNLSVSVENLCTVIWTWDPPEGASPNCTLRYFSHFDNKQDKKIAPETHRSKEVPLNERICLQVGSQCSTNESDNPSILVEKCTPPPEGDPESAVTELQCVWHNLSYMKCTWLPGRNTSPDTNYTLYYWHSSLGKILQCEDIYREGQHIGCSFALTNLKDSSFEQHSVQIVVKDNAGKIRPSFNIVPLTSHVKPDPPHIKRLFFQNGNLYVQWKNPQNFYSRCLSYQVEVNNSQTETNDIFYVEEAKCQNSEFEGNLEGTICFMVPGVLPDTLNTVRIRVRTNKLCYEDDKLWSNWSQAMSI

SEQ ID NO: 33 - protein, 184 aa, synthetic construct
  REGION 1..184: note = Canine mini-IL4R ECD
KVLHEPSCFSDYISTSVCQWKMDHPTNCSAELRLSYQLDFMGSENHTCVPENREDSVCVCSMPIDDAVEADVYQLDLWAGQQLLWSGSFQPSKHVKPRTPGNLTVHPNISHTWLLMWTNPYPTENHLHSELTYMVNVSNDNDPEDFKVYNVTYMGPTLRLAASTLKSGASYSARVRAWAQTYNS

SEQ ID NO: 34 - protein, 305 aa, synthetic construct
  REGION 1..305: note = Feline mini-IL13R ECD
QPPVTNLSVSVENLCTVIWTWDPPEGASPNCTLRYFSHFDNKQDKKIAPETHRSKEVPLNERICLQVGSQCSTNESDNPSILVEKCTPPPEGDPESAVTELQCVWHNLSYMKCTWLPGRNTSPDTNYTLYYWHSSLGKILQCENIYREGQHIGCSFALTNLKDSSFEQHSVQIVVKDNAGKIRPSFNIVPLTSHVKPDPPHIKRLFFQNGNLYVQWKNPQNFYSRCLSYQVEVNNSQTETHDIFYVEEAKCQNSEFEGNLEGTICFMVPGILPDTLNTVRIRVRTNKLCYEDDRLWSNWSQAMSI

SEQ ID NO: 35 - protein, 266 aa, synthetic construct
  REGION 1..266: note = Feline mini-IL4R ECD
KVLRAPTCFSDYFSTSVCQWNMDAPTNCSAELRLSYQLNFMGSENRTCVPENGEGAACACSMLMDDFVEADVYQLHLWAGTQLLWSGSFKPSSHVKPRAPGNLTVHPNVSHTWLLRWSNPYPPENHLHAELTYMVNISSEDDPTDVSVCASGFLCHLLGLRRVETGAPGARLPPWLCAPRPRRVPGSQCAVISCCRWVLIALTSRGGRWRLTPGLRSQTRYVSVAEGLFGATPRVLCPGTQAGLASAAREQMSPDPSAFHSIDYEP

SEQ ID NO: 36 - protein, 305 aa, synthetic construct
  REGION 1..305: note = Equine mini-IL13R ECD
QPPVTNLSVSVENLCTVIWTWNPPEGVSPNCSLWYFSHFGNKQDKKIAPETHRSKEVPLNERICLQVGSQCSTNESDNPSILVEKCISPPEGDPESAVTELQCVWHNLSYMKCTWLPGKNASPDTNYTLYYWHSSLGKILQCEDIYREGQHIGCSFALTEVKDSIFEQHSVQIMVKDNAGKIRPFFNIVPLTSHVKPDPPHIKKLFFQNGDLYVQWKNPQNFYSRCLSYQVEVNNSQTETRDIFSVEEAKCQNPEFEGDLEGTICFMVPGVLPDTVNTVRIRVKTNKLCYEDDKLWSNWSQAMSI

SEQ ID NO: 37 - protein, 201 aa, synthetic construct
  REGION 1..201: note = Equine mini-IL4R ECD
KVLHLTACFSDYISASTCEWKMDRPTNCSAQLRLSYQLNDEFSDNLTCIPENREDEVCVCRMLMDNIVSEDVYELDLWAGNQLLWNSSFKPSRHVKPRAPQNLTVHAISHTWLLTWSNPYPLKNHLWSELTYLVNISKEDDPTDFKIYNVTYMDPTLRVTASTLKSRATYSARVKARAQNYNSTWSEWSPSTTWHNYYEQP

SEQ ID NO: 38 - protein, 4 aa, synthetic construct
  REGION 1..4: note = GS linker
GSGS

SEQ ID NO: 39 - protein, 6 aa, synthetic construct
  REGION 1..6: note = GS linker
GSGSGS

SEQ ID NO: 40 - protein, 6 aa, synthetic construct
  REGION 1..6: note = GS linker
GGSGGS

SEQ ID NO: 41 - protein, 9 aa, synthetic construct
  REGION 1..9: note = GS linker
GGSGGSGGS

SEQ ID NO: 42 - protein, 4 aa, synthetic construct
  REGION 1..4: note = GS linker
GGGS

SEQ ID NO: 43 - protein, 8 aa, synthetic construct
  REGION 1..8: note = GS linker
GGGSGGGS

SEQ ID NO: 44 - protein, 12 aa, synthetic construct
  REGION 1..12: note = GS linker
GGGSGGGSGGGS

SEQ ID NO: 45 - protein, 6 aa, synthetic construct
  REGION 1..6: note = GS linker
GSSGSS

SEQ ID NO: 46 - protein, 9 aa, synthetic construct
  REGION 1..9: note = GS linker
GSSGSSGSS

SEQ ID NO: 47 - protein, 4 aa, synthetic construct
  REGION 1..4: note = GS linker
GGSS

SEQ ID NO: 48 - protein, 8 aa, synthetic construct
  REGION 1..8: note = GS linker
GGSSGGSS

SEQ ID NO: 49 - protein, 12 aa, synthetic construct
  REGION 1..12: note = GS linker
GGSSGGSSGGSS

(19) United States
(12) Patent Application Publication
(10) Pub. No.: US 2024/0270820 A1
(43) Pub. Date: Aug. 15, 2024

(54) IL4/IL13 RECEPTOR MOLECULE FOR VETERINARY USE

(71) Applicant: Elanco US Inc., Greenfield, IN (US)

(72) Inventors: Hangjun ZHAN, Burlingame, CA (US); Lam NGUYEN, Burlingame, CA (US); Fawn QIAN, Burlingame, CA (US); Shyr Jiann LI, Burlingame, CA (US)

(21) Appl. No.: 18/617,972

(22) Filed: Mar. 27, 2024

Related U.S. Application Data
(62) Division of application No. 16/605,356, filed on Oct. 15, 2019, now Pat. No. 11,970,526.
(60) Provisional application No. 62/488,509, filed on Apr. 21, 2017.

(51) Int. Cl. C07K 14/715; A61K 38/17; A61K 45/06; G01N 33/68

(52) U.S. Cl. CPC C07K 14/7155; A61K 38/1793; G01N 33/6869; A61K 45/06; C07K 2319/035; C07K 2319/30

(57) ABSTRACT

Provided are various embodiments relating to IL13R/IL4R contiguous polypeptides derived from companion animal species and that bind to IL13 and/or IL4. Such contiguous polypeptides can be used in methods to treat IL13 and/or IL4-induced conditions in companion animals, such as canines, felines, and equines.

### CROSS-REFERENCE TO RELATED APPLICATIONS

This application is a Divisional application of U.S. application Ser. No. 16/605,356, filed Oct. 15, 2019, which is a national stage of International Application No. PCT/US2018/028507, filed Apr. 20, 2018, which claims the benefit of priority of U.S. Provisional Application No. 62/488,509, filed Apr. 21, 2017, the entire contents of each of which are incorporated herein by reference.

### REFERENCE TO SEQUENCE LISTING SUBMITTED AS AN XML FILE (.xml)

A Sequence Listing is submitted herewith as an XML file named “2920951-452002_Sequence_Listing_ST26” created on Mar. 19, 2024 and having a size of 75,204 bytes as permitted under 37 C.F.R. § 1.821 (c). The material in the aforementioned file is hereby incorporated by reference in its entirety.

### FIELD

This present disclosure relates to contiguous polypeptides comprising interleukin 4 receptor and interleukin 13 receptor fragments derived from companion animal species that bind to IL4 and/or IL13 of a companion animal species, for example, canine IL4 and canine IL13. This invention also relates to methods of using the contiguous polypeptides, for example, for treating IL4 and/or IL13-induced conditions or reducing IL4 and/or IL13 signaling activity in cells, for instance in companion animals, such as canines, felines, and equines.

### BACKGROUND

Interleukin 4 (IL4) is a cytokine promoting differentiation of naïve helper T cells to Th2 cells. Interleukin 13 (IL13) has similar effects on immune cells. Both IL4 and IL13 play important roles in T cell-mediated immune responses that are directly associated with allergy, for example, atopic dermatitis and asthma. It is generally understood that IL4 can form a signaling complex either with heterodimeric receptors IL4 receptor subunit alpha (IL4R) and γc or IL4R and IL13 receptor subunit alpha-1 (IL13R). IL13 can form a signaling complex with heterodimeric receptors IL4Ra and IL13Ra1. Extracellular domains of IL4Ra or IL13Ra1 may bind to IL4 and/or IL13 and reduce the free concentrations of the cytokines, thus diminishing the clinical signs and symptoms associated with dermatitis, asthma and other disorders.

Companion species animals, such as cats, dogs, and horses, suffer from many allergic diseases similar to human allergic diseases, including atopic dermatitis and asthma. There remains a need, therefore, for methods and compounds that can be used specifically to bind companion animal IL4 and/or IL13 for treating IL4/IL13-induced conditions and for reducing IL4/IL13 signaling activity.

### SUMMARY

In some embodiments, an IL13R/IL4R contiguous polypeptide is provided that comprises an extracellular domain of an IL13R polypeptide and an extracellular domain of an IL4R polypeptide, wherein the IL13R and IL4R polypeptides are derived from a companion animal species.

In some embodiments, an IL13R/IL4R contiguous polypeptide comprises formula (I) IL13R-L1-IL4R-L2-FP or formula (II) IL4R-L1-IL13R-L2-FP, wherein:
a. IL13R is an extracellular domain of an IL13R polypeptide derived from the companion animal species,
b. IL4R is an extracellular domain of an IL4R polypeptide derived from the companion animal species,
c. L1 is a first optional linker,
d. L2 is a second optional linker, and
e. FP is a fusion partner.

In some embodiments, an IL13R/IL4R contiguous polypeptide binds to IL13 of a companion animal species with a dissociation constant (Kd) of less than 5×10⁻⁶ M, less than 1×10⁻⁶ M, less than 5×10⁻⁷ M, less than 1×10⁻⁷ M, less than 5×10⁻⁸ M, less than 1×10⁻⁸ M, less than 5×10⁻⁹ M, less than 1×10⁻⁹ M, less than 5×10⁻¹⁰ M, less than 1×10⁻¹⁰ M, less than 5×10⁻¹¹ M, less than 1×10⁻¹¹ M, less than 5×10⁻¹² M, or less than 1×10⁻¹² M, as measured by biolayer interferometry.

In some embodiments, an IL13R/IL4R contiguous polypeptide binds to IL4 of a companion animal species with a dissociation constant (Kd) of less than 5×10⁻⁶ M, less than 1×10⁻⁶ M, less than 5×10⁻⁷ M, less than 1×10⁻⁷ M, less than 5×10⁻⁸ M, less than 1×10⁻⁸ M, less than 5×10⁻⁹ M, less than 1×10⁻⁹ M, less than 5×10⁻¹⁰ M, less than 1×10⁻¹⁰ M, less than 5×10⁻¹¹ M, less than 1×10⁻¹¹ M, less than 5×10⁻¹² M, or less than 1×10⁻¹² M, as measured by biolayer interferometry.

In some embodiments, an IL13R/IL4R contiguous polypeptide reduces IL13 and/or IL4 signaling in the companion animal species.

In some embodiments, the companion animal species is canine, feline, or equine.

In some embodiments, the extracellular domain of a IL13R polypeptide is at least 85% identical to the amino acid sequence of SEQ ID NO: 22, SEQ ID NO: 24, SEQ ID NO: 26, SEQ ID NO: 32, SEQ ID NO: 34, or SEQ ID NO: 36. In some embodiments, the extracellular domain of the IL13R polypeptide is at least 90% identical to the amino acid sequence of SEQ ID NO: 22, SEQ ID NO: 24, SEQ ID NO: 26, SEQ ID NO: 32, SEQ ID NO: 34, or SEQ ID NO: 36. In some embodiments, the extracellular domain of the IL13R polypeptide is at least 95% identical to the amino acid sequence of SEQ ID NO: 22, SEQ ID NO: 24, SEQ ID NO: 26, SEQ ID NO: 32, SEQ ID NO: 34, or SEQ ID NO: 36. In some embodiments, the extracellular domain of the IL13R polypeptide is at least 98% identical to the amino acid sequence of SEQ ID NO: 22, SEQ ID NO: 24, SEQ ID NO: 26, SEQ ID NO: 32, SEQ ID NO: 34, or SEQ ID NO: 36.

In some embodiments, the extracellular domain of the IL13R polypeptide comprises a cysteine at a position corresponding to position 18 of SEQ ID NO: 22, corresponding to position 18 of SEQ ID NO: 24, or corresponding to position 18 of SEQ ID NO: 26. In some embodiments, the extracellular domain of an IL13R polypeptide comprises a cysteine at position 18 of SEQ ID NO: 22, at position 18 of SEQ ID NO: 24, at position 18 of SEQ ID NO: 26, at position 15 of SEQ ID NO: 32, at position 15 of SEQ ID NO: 34, or at position 15 of SEQ ID NO: 36. In some embodiments, the extracellular domain of the IL13R polypeptide comprises an amino acid sequence selected from SEQ ID NO: 32, SEQ ID NO: 34, and SEQ ID NO: 36. In some embodiments, the extracellular domain of the IL13R polypeptide comprises an amino acid sequence selected from SEQ ID NO: 22, SEQ ID NO: 24, and SEQ ID NO: 26.

In some embodiments, the extracellular domain of the IL4R polypeptide is at least 85% identical to the amino acid sequence of SEQ ID NO: 23, SEQ ID NO: 25, SEQ ID NO: 27, SEQ ID NO: 33, SEQ ID NO: 35, or SEQ ID NO: 37. In some embodiments, the extracellular domain of the IL4R polypeptide is at least 90% identical to the amino acid sequence of SEQ ID NO: 23, SEQ ID NO: 25, SEQ ID NO: 27, SEQ ID NO: 33, SEQ ID NO: 35, or SEQ ID NO: 37. In some embodiments, the extracellular domain of the IL4R polypeptide is at least 95% identical to the amino acid sequence of SEQ ID NO: 23, SEQ ID NO: 25, SEQ ID NO: 27, SEQ ID NO: 33, SEQ ID NO: 35, or SEQ ID NO: 37. In some embodiments, the extracellular domain of the IL4R polypeptide is at least 98% identical to the amino acid sequence of SEQ ID NO: 23, SEQ ID NO: 25, SEQ ID NO: 27, SEQ ID NO: 33, SEQ ID NO: 35, or SEQ ID NO: 37. In some embodiments, the extracellular domain of the IL4R polypeptide comprises an amino acid sequence selected from SEQ ID NO: 33, SEQ ID NO: 35, and SEQ ID NO: 37. In some embodiments, the extracellular domain of the IL4R polypeptide comprises an amino acid sequence selected from SEQ ID NO: 23, SEQ ID NO: 25, and SEQ ID NO: 27.

In some embodiments, L1 and L2, if present, each independently comprises an amino acid sequence selected from G, GG, GGG, S, SS, SSS, GS, GSGS (SEQ ID NO: 38), GSGSGS (SEQ ID NO: 39), GGS, GGSGGS (SEQ ID NO: 40), GGSGGSGGS (SEQ ID NO: 41), GGGS (SEQ ID NO: 42), GGGSGGGS (SEQ ID NO: 43), GGGSGGGSGGGS (SEQ ID NO: 44), GSS, GSSGSS (SEQ ID NO: 45), GSSGSSGSS (SEQ ID NO: 46), GGSS (SEQ ID NO: 47), GGSSGGSS (SEQ ID NO: 48), and GGSSGGSSGGSS (SEQ ID NO: 49).

In some embodiments, the fusion partner is selected from an Fc, albumin, and an albumin binding fragment. In some embodiments, the Fc is (a) a canine heavy chain constant region selected from an IgG-A, IgG-B, IgG-C, and IgG-D constant region; (b) a feline heavy chain constant region selected from an IgG1, IgG2a, and IgG2b constant region; or (c) an equine heavy chain constant region selected from an IgG1, IgG2, IgG3, IgG4, IgG5, IgG6 and IgG7 constant region.

In some embodiments, an IL13R/IL4R contiguous polypeptide comprises the sequence selected from SEQ ID NO: 13, SEQ ID NO: 14, SEQ ID NO: 15, SEQ ID NO: 16, SEQ ID NO: 17, SEQ ID NO 18, SEQ ID NO: 19, SEQ ID NO: 20, SEQ ID NO: 21, SEQ ID NO: 28, SEQ ID NO: 29, SEQ ID NO: 30, and SEQ ID NO: 31.

In some embodiments, an isolated nucleic acid is provided, which encodes an IL13R/IL4R contiguous polypeptide described herein above. In some embodiments, a host cell is provided, which comprises a nucleic acid encoding an IL13R/IL4R contiguous polypeptide described herein above. In some embodiments, a method of producing an IL13R/IL4R contiguous polypeptide is provided, which comprises culturing such a host cell comprising a nucleic acid encoding an IL13R/IL4R contiguous polypeptide described herein above and isolating the contiguous polypeptide. In some embodiments, a pharmaceutical composition is provided, which comprises an IL13R/IL4R contiguous polypeptide described herein and a pharmaceutically acceptable carrier.

In some embodiments, methods of treating a companion animal species having an IL13 and/or IL4-induced condition are provided, comprising administering to the companion animal species a therapeutically effective amount of an IL13R/IL4R contiguous polypeptide described herein or a pharmaceutical composition comprising an IL13R/IL4R contiguous polypeptide described herein. In some embodiments, the companion animal species is canine, feline, or equine. In some embodiments, the IL13 and/or IL4-induced condition is a pruritic or allergic condition, such as atopic dermatitis, pruritus, asthma, psoriasis, scleroderma, or eczema.

In some embodiments, the IL13R/IL4R contiguous polypeptide or the pharmaceutical composition is administered parenterally. In some embodiments, the IL13R/IL4R contiguous polypeptide or the pharmaceutical composition is administered by an intramuscular route, an intraperitoneal route, an intracerebrospinal route, a subcutaneous route, an intra-arterial route, an intrasynovial route, an intrathecal route, or an inhalation route.

In some embodiments, the method further comprises administering a Jak inhibitor, a PI3K inhibitor, an AKT inhibitor, or a MAPK inhibitor. In some embodiments, the method further comprises administering one or more antibodies selected from an anti-IL17 antibody, an anti-IL31 antibody, an anti-TNFα antibody, an anti-CD20 antibody, an anti-CD19 antibody, an anti-CD25 antibody, an anti-IL4 antibody, an anti-IL13 antibody, an anti-IL23 antibody, an anti-IgE antibody, an anti-CD11α antibody, anti-IL6R antibody, anti-α4-Integrin antibody, an anti-IL12 antibody, an anti-IL1B antibody, and an anti-BlyS antibody.

In some embodiments, methods of reducing IL13 and/or IL4 signaling activity in a cell are provided, comprising exposing the cell to an IL13R/IL4R contiguous polypeptide or pharmaceutical composition described herein under conditions permissive for binding of the contiguous polypeptide to IL13 and/or IL4, thereby (a) reducing binding of IL/4 and/or IL-13 to native IL13 receptor and/or native IL-4 receptor and reducing IL13- and/or IL-4-mediated signaling. In some embodiments, the cell is exposed to the IL13R/IL4R contiguous polypeptide or the pharmaceutical composition ex vivo. In some embodiments, the cell is exposed to the contiguous polypeptide or the pharmaceutical composition in vivo. In some embodiments, the cell is a canine cell, a feline cell, or an equine cell.

In some embodiments, methods for detecting IL13 or IL4 in a sample from a companion animal species are provided, comprising contacting the sample with an IL13R/IL4R contiguous polypeptide or a pharmaceutical composition described herein under conditions permissive for binding of the contiguous polypeptide to IL13 and/or IL4, and detecting whether a complex is formed between the contiguous polypeptide and IL13 and/or IL4 in the sample. In some embodiments, the sample is a biological sample obtained from a canine, a feline, or an equine.

### BRIEF DESCRIPTION OF THE DRAWINGS

FIG. 1 is a graph of canine IL4R-IL13R-Fc sequential binding to canine IL4 and IL13 or canine IL13 and IL4 using concentrations of 30 μg/mL of IL4 and IL13 in PBS.

FIG. 2 is a graph of canine IL13R-IL4R-Fc sequential binding to canine IL4 and IL13 or canine IL13 and IL4 using concentrations of 30 μg/mL of IL4 and IL13 in PBS.

FIG. 3 is a graph of canine IL4R-IL13R-Fc neutralizing canine IL4 activity in a TF1 cell proliferation assay. Canine IL4 (50 ng/ml or 3.85 nM) was used in the assay.

### DESCRIPTION OF THE SEQUENCES

Table 1 provides a listing of certain sequences referenced herein.

TABLE 1
Description of the Sequences

| SEQ ID | | |
|---|---|---|
| NO: | SEQUENCE | DESCRIPTION |
| 1 | MGLTSQLIPTLVCLLALTSTFVHGHNFNITIKEI | Canis lupus |
| | IKMLNILTARNDSCMELTVDVFTAPKNTSDKEI | interleukin-4 precursor |
| | FCRAATVLRQIYTHNCSNRYLRGLYRNLSSMA | |
| | NKTCSMNEIKKSTLKDFLERLKVIMQKKYYRH | |
| 2 | MDLTSQLIPALVCLLAFTSTFVHGQNFNNTLK | Felis catus |
| | EIIKTLNILTARNDSCMELTMDVLAAPKNTSD | interleukin-4 precursor |
| | KEIFCRATTVLRQIYTHEINCSTKFLKGLDRNLS | |
| | SMANRTCSVNEVKKCTLKDFLERLKAIMQKK | |
| | YSKH | |
| 3 | MGLTYQLIPALVCLLACTSNFIQGCKYDITLQE | Equus caballus interleukin-4 |
| | IIKTLNNLTDGKGKNSCMELTVADAFAGPKNT | precursor |
| | DGKEICRAAKVLQQLYKRHDRSLIKECLSGLD | |
| | RNLKGMANGTCCTVNEAKKSTLKDFLERLKTI | |
| | MKEKYSKC | |
| 4 | MALWLTVVIALTCLGGLASPSPVTPSPTLKELI | Canis lupus |
| | EELVNITQNQASLCNGSMVWSVNLTAGMYCA | interleukin-13 precursor |
| | ALESLINVSDCSAIQRTQRMLKALCSQKPAAG | |
| | QISSERSRDTKIEVIQLVKNLLTYVRGVYRHGN | |
| | FR | |
| 5 | MWFLDSTRQSGDQGGRRHTWPIKATARGQG | Felis catus |
| | HKPLSLGQPTCPLLAPPVLALGSMALWLTVVI | interleukin-13 precursor |
| | ALTCLGGLASPGPHSRRELKELIEELVNITQNQ | |
| | VSLCNGSMVWSVNLTTGMYCAALESLINVSD | |
| | CTAIQRTQRMLKALCTQKPSAGQTASERSRDT | |
| | KIEVIQLVKNLLNHLRRNFRHGNFK | |
| 6 | MALWLTAVIALACLGGLASPAPLPSSMALKEL | Equus caballus interleukin- |
| | IKELVNITQNQAPLCNGSMVWSVNLTADTYC | 13 precursor |
| | RALESLSNVSTCSAIQNTRKMLTKLCPHQLSA | |
| | GQVSSERARDTKIEVIVLVKDLLKNLRKIFHGG | |
| | KHVDA | |
| 7 | MGRLCSGLTFPVSCLVLVWVASSGSVKVLHE | Canis lupus |
| | PSCFSDYISTSVCQWKMDHPTNCSAELRLSYQ | interleukin-4 receptor |
| | LDFMGSENHTCVPENREDSVCVCSMPIDDAVE | subunit alpha |
| | ADVYQLDLWAGQQLLWSGSFQPSKHVKPRTP | |
| | GNLTVHPNISHTWLLMWTNPYPTENHLHSELT | |
| | YMVNVSNDNDPEDFKVYNVTYMGPTLRLAA | |
| | STLKSGASYSARVRAWAQTYNSTWSDWSPST | |
| | TWLNYYEPWEQHLPLGVSISCLVILAICLSCYF | |
| | SIIKIKKGWWDQIPNPAHSPLVAIVIQDSQVSL | |
| | WGKRSRGQEPAKCPHWKTCLTKLLPCLLEHG | |
| | LGREEESPKTAKNGPLQGPGKPAWCPVEVSKT | |
| | ILWPESISVVQCVELSEAPVDNEEEEEVEEDKR | |
| | SLCPSLEGSGGSFQEGREGIVARLTESLFLDLL | |
| | GGENGGFCPQGLEESCLPPPSGSVGAQMPWA | |
| | QFPRAGPRAAPEGPEQPRRPESALQASPTQSA | |
| | GSSAFPEPPPVVTDNPAYRSFGSFLGQSSDPGD | |
| | GDSDPELADRPGEADPGIPSAPQPPEPPAALQP | |
| | EPESWEQILRQSVLQHRAAPAPGPGPGSGYRE | |
| | FTCAVKQGSAPDAGGPGFGPSGEAGYKAFCSL | |
| | LPGGATCPGTSGGEAGSGEGGYKPFQSLTPGC | |
| | PGAPTPVPVPLFTFGLDTEPPGSPQDSLGAGSS | |
| | PEHLGVEPAGKEEDSRKTLLAPEQATDPLRDD | |
| | LASSIVYSALTCHLCGHLKQWHDQEERGKAHI | |
| | VPSPCCGCCCGDRSSLLLSPLRAPNVLPGGVLL | |
| | EASLSPASLVPSGVSKEGKSSPFSQPASSSAQSS | |
| | SQTPKKLAVLSTEPTCMSAS | |
| 8 | MGRLCSGLTFPVSCLILMWAAGSGSVKVLRA | Felis catus |
| | PTCFSDYFSTSVCQWNMDAPTNCSAELRLSYQ | interleukin-4 receptor |
| | LNFMGSENRTCVPENGEGAACACSMLMDDFV | subunit alpha |
| | EADVYQLHLWAGTQLLWSGSFKPSSHVKPRA | |
| | PGNLTVHPNVSHTWLLRWSNPYPPENHLHAE | |
| | LTYMVNISSEDDPTDVSVCASGFLCHLLGLRR | |
| | VETGAPGARLPPWLCAPRPRRVPGSQCAVISC | |
| | CRWVLIALTSRGGRWRLTPGLRSQTRYVSVAE | |
| | GLFGATPRVLCPGTQAGLASAAREQMSPDPSA | |
| | FHSIDYEPWEQHLPLGVSISCLVILAVCLSCYLS | |
| | VIKIKKEWWDQIPNPAHSHLVAIVIQDPQVSL | |
| | WGKRSRGQEPAKCPHWKTCLRKLLPCLLEHG | |
| | MERKEDPSKIARNGPSQCSGKSAWCPVEVSKT | |
| | ILWPESISVVRCVELLEAPVESEEEEEEEEDKGS | |
| | FCPSPVNLEDSFQEGREGIAARLTESLFMDLLG | |
| | VEKGGFGPQGSLESWFPPPSGSAGAQMPWAE | |
| | FPGPGPQEASPQGKEQPFDPRSDPLATLPQSPA | |
| | SPTFPETPPVVTDNPAYRSFGTFQGRSSGPGEC | |
| | DSGPELAGRLGEADPGIPAAPQPSEPPSALQPE | |
| | AETWEQILRQRVLQHRGAPAPAPGSGYREFVC | |
| | AVRQGSTQDSGVGDFGPSEEAGYKAFSSLLTS | |
| | GAVCPESGGEAGSGDGGYKPFQSLTPGCPGAP | |
| | APVPVPLFTFGLDAEPPHCPQDSPLPGSSPEPA | |
| | GKAQDSHKTPPAPEQAADPLRDDLASGIVYSA | |
| | LTCHLCGHLKQCHGQEEGGEAHPVASPCCGC | |
| | CCGDRSSPLVSPLRAPDPLPGGVPLEASLSPAS | |
| | PAPLAVSEEGPPSLCFQPALSHAHSSSQTPKKV | |
| | AMLSPEPTCTMAS | |
| 9 | MGCLCPGLTLPVSCLILVWAAGSGSVKVLHLT | Equus caballus |
| | ACFSDYISASTCEWKMDRPTNCSAQLRLSYQL | interleukin-4 receptor |
| | NDEFSDNLTCIPENREDEVCVCRMLMDNIVSE | subunit alpha |
| | DVYELDLWAGNQLLWNSSFKPSRHVKPRAPQ | |
| | NLTVHAISHTWLLTWSNPYPLKNHLWSELTYL | |
| | VNISKEDDPTDFKIYNVTYMDPTLRVTASTLK | |
| | SRATYSARVKARAQNYNSTWSEWSPSTTWHN | |
| | YYEQPLEQRLPLGVSISCVVILAICLSCYFSIIKI | |
| | KKEWWDQIPNPAHSPLVAIVLQDSQVSLWGK | |
| | QSRGQEPAKCPRWKTCLTKLLPCLLEHGLQKE | |
| | EDSSKTVRNGPFQSPGKSAWHTVEVNHTILRP | |
| | EIISVVPCVELCEAQVESEEEEVEEDRGSFCPSP | |
| | ESSGSGFQEGREGVAARLTESLFLGLLGAENG | |
| | ALGESCLLPPLGSAHMPWARISSAGPQEAASQ | |
| | GEEQPLNPESNPLATLTQSPGSLAFTEAPAVVA | |
| | DNPAYRSFSNSLSQPRGPGELDSDPQLAEHLG | |
| | QVDPSIPSAPQPSEPPTALQPEPETWEQMLRQS | |
| | VLQQGAAPAPASAPTGGYREFAQAVKQGGGA | |
| | AGSGPSGEAGYKAFSSLLAGSAVCPGQSGVEA | |
| | SSGEGGYRPYESPDPGAPAPVPVPLFTFGLDVE | |
| | PPHSPQNSLLPGGSPELPGPEPTVKGEDPRKPL | |
| | LSAQQATDSLRDDLGSGIVYSALTCHLCGHLK | |
| | QCHGQEEHGEAHTVASPCCGCCCGDRSSPPVS | |
| | PVRALDPPPGGVPLEAGLSLASLGSLGLSEERK | |
| | PSLFFQPAPGNAQSSSQTPLTVAMLSTGPTCTS | |
| | AS | |
| 10 | MERPARLCGLWALLLCAAGGRGGGVAAPTET | Canis lupus |
| | QPPVTNLSVSVENLCTVIWTWDPPEGASPNCT | interleukin-13 receptor |
| | LRYFSHFDNKQDKKIAPETHRSKEVPLNERICL | subunit alpha-1 |
| | QVGSQCSTNESDNPSILVEKCTPPPEGDPESAV | |
| | TELQCVWHNLSYMKCTWLPGRNTSPDTNYTL | |
| | YYWHSSLGKILQCEDIYREGQHIGCSFALTNL | |
| | KDSSFEQHSVQIVVKDNAGKIRPSFNIVPLTSH | |
| | VKPDPPHIKRLFFQNGNLYVQWKNPQNFYSR | |
| | CLSYQVEVNNSQTETNDIFYVEEAKCQNSEFE | |
| | GNLEGTICFMVPGVLPDTLNTVRIRVRTNKLC | |
| | YEDDKLWSNWSQAMSIGENTDPTFYITMLLA | |
| | TPVIVAGAIIVLLLYLKRLKIIIFPPIPDPGKIFKE | |
| | MFGDQNDDTLHWRKYDIYEKQTKEETDSVVL | |
| | IENLKKASQ | |
| 11 | MMTKCSSDRNVFKRKWFLFPASQYTFRPIHQ | Felis catus |
| | ARPCEVPAVHLEPSPPWEVGLGLLNLESEFRK | interleukin-13 receptor |
| | LGLRGRRLAAAPPDSRAEAASQTQPPVTNLSV | subunit alpha-1 |
| | SVENLCTVIWTWDPPEGASPNCTLRYFSHFDN | |
| | KQDKKIAPETHRSKEVPLNERICLQVGSQCSTN | |
| | ESDNPSILVEKCTPPPEGDPESAVTELQCVWHN | |
| | LSYMKCTWLPGRNTSPDTNYTLYYWHSSLGK | |
| | ILQCENIYREGQHIGCSFALTNLKDSSFEQHSV | |
| | QIVVKDNAGKIRPSFNIVPLTSHVKPDPPHIKRL | |
| | FFQNGNLYVQWKNPQNFYSRCLSYQVEVNNS | |
| | QTETHDIFYVEEAKCQNSEFEGNLEGTICFMVP | |
| | GILPDTLNTVRIRVRTNKLCYEDDRLWSNWSQ | |
| | AMSIGENTDPTFYITMLLATPVIVAGAIIVLLLY | |
| | LKRLKIIIFPPIPDPGKIFKEMFGDQNDDSLHWK | |
| | KYDIYEKQTKEETDSVVLIENASQ | |
| 12 | MYFLCLIWTESQPPVTNLSVSVENLCTVIWTW | Equus caballus |
| | NPPEGVSPNCSLWYFSHFGNKQDKKIAPETHR | interleukin-13 receptor |
| | SKEVPLNERICLQVGSQCSTNESDNPSILVEKCI | subunit alpha-1 |
| | SPPEGDPESAVTELQCVWHNLSYMKCTWLPG | |
| | KNASPDTNYTLYYWHSSLGKILQCEDIYREGQ | |
| | HIGCSFALTEVKDSIFEQHSVQIMVKDNAGKIR | |
| | PFFNIVPLTSHVKPDPPHIKKLFFQNGDLYVQW | |
| | KNPQNFYSRCLSYQVEVNNSQTETRDIFSVEE | |
| | AKCQNPEFEGDLEGTICFMVPGVLPDTVNTVR | |
| | IRVKTNKLCYEDDKLWSNWSQAMSIGKKADP | |
| | TFYIAMLLIIPVIVAGAIIVLLLYLKRLKIIMFPPI | |
| | PDPGKIFKEMFGDQNDDTLHWKKYDIYEKQT | |
| | KEETDSVVLIENLKRASQ | |
| 13 | TETQPPVTNLSVSVENLCTVIWTWDPPEGASP | Canis lupus IL13R-IL4R-Fc |
| | NCTLRYFSHFDNKQDKKIAPETHRSKEVPLNE | (without signal sequence) |
| | RICLQVGSQCSTNESDNPSILVEKCTPPPEGDPE | |
| | SAVTELQCVWHNLSYMKCTWLPGRNTSPDTN | |
| | YTLYYWHSSLGKILQCEDIYREGQHIGCSFALT | |
| | NLKDSSFEQHSVQIVVKDNAGKIRPSFNIVPLT | |
| | SHVKPDPPHIKRLFFQNGNLYVQWKNPQNFYS | |
| | RCLSYQVEVNNSQTETNDIFYVEEAKCQNSEF | |
| | EGNLEGTICFMVPGVLPDTLNTVRIRVRTNKL | |
| | CYEDDKLWSNWSQAMSIGENTDPTGGGSGSG | |
| | SVKVLHEPSCFSDYISTSVCQWKMDHPTNCSA | |
| | ELRLSYQLDFMGSENHTCVPENREDSVCVCS | |
| | MPIDDAVEADVYQLDLWAGQQLLWSGSFQPS | |
| | KHVKPRTPGNLTVHPNISHTWLLMWTNPYPT | |
| | ENHLHSELTYMVNVSNDNDPEDFKVYNVTY | |
| | MGPTLRLAASTLKSGASYSARVRAWAQTYNS | |
| | TWSDWSPSTTWLNYYEPKRENGRVPRPPDCP | |
| | KCPAPEMLGGPSVFIFPPKPKDTLLIARTPEVT | |
| | CVVVDLDPEDPEVQISWFVDGKQMQTAKTQP | |
| | REEQFNGTYRVVSVLPIGHQDWLKGKQFTCK | |
| | VNNKALPSPIERTISKARGQAHQPSVYVLPPSR | |
| | EELSKNTVSLTCLIKDFFPPDIDVEWQSNGQQE | |
| | PESKYRTTPPQLDEDGSYFLYSKLSVDKSRWQ | |
| | RGDTFICAVMHEALHNHYTQESLSHSPGK | |
| 14 | SGSVKVLHEPSCFSDYISTSVCQWKMDHPTNC | Canis lupus IL4R-IL13R- |
| | SAELRLSYQLDFMGSENHTCVPENREDSVCVC | IgGA (without signal |
| | SMPIDDAVEADVYQLDLWAGQQLLWSGSFQP | sequence) |
| | SKHVKPRTPGNLTVHPNISHTWLLMWTNPYPT | |
| | ENHLHSELTYMVNVSNDNDPEDFKVYNVTY | |
| | MGPTLRLAASTLKSGASYSARVRAWAQTYNS | |
| | TWSDWSPSTTWLNYYEPGGGSGTETQPPVTN | |
| | LSVSVENLCTVIWTWDPPEGASPNCTLRYFSH | |
| | FDNKQDKKIAPETHRSKEVPLNERICLQVGSQ | |
| | CSTNESDNPSILVEKCTPPPEGDPESAVTELQC | |
| | VWHNLSYMKCTWLPGRNTSPDTNYTLYYWH | |
| | SSLGKILQCEDIYREGQHIGCSFALTNLKDSSFE | |
| | QHSVQIVVKDNAGKIRPSFNIVPLTSHVKPDPP | |
| | HIKRLFFQNGNLYVQWKNPQNFYSRCLSYQV | |
| | EVNNSQTETNDIFYVEEAKCQNSEFEGNLEGTI | |
| | CFMVPGVLPDTLNTVRIRVRTNKLCYEDDKL | |
| | WSNWSQAMSIGENTDPTFNECRCTDTPPCPVP | |
| | EPLGGPSVLIFPPKPKDILRITRTPEVTCVVLDL | |
| | GREDPEVQISWFVDGKEVHTAKTQSREQQFN | |
| | GTYRVVSVLPIEHQDWLTGKEFKCRVNHIDLP | |
| | SPIERTISKARGRAHKPSVYVLPPSPKELSSSDT | |
| | VSITCLIKDFYPPDIDVEWQSNGQQEPERKHR | |
| | MTPPQLDEDGSYFLYSKLSVDKSRWQQGDPF | |
| | TCAVMHETLQNHYTDLSLSHSPGK | |
| 15 | SGSVKVLHEPSCFSDYISTSVCQWKMDHPTNC | Canis lupus |
| | SAELRLSYQLDFMGSENHTCVPENREDSVCVC | IL4R-IL13R-IgGB |
| | SMPIDDAVEADVYQLDLWAGQQLLWSGSFQP | (without signal sequence) |
| | SKHVKPRTPGNLTVHPNISHTWLLMWTNPYPT | |
| | ENHLHSELTYMVNVSNDNDPEDFKVYNVTY | |
| | MGPTLRLAASTLKSGASYSARVRAWAQTYNS | |
| | TWSDWSPSTTWLNYYEPGGGSGTETQPPVTN | |
| | LSVSVENLCTVIWTWDPPEGASPNCTLRYFSH | |
| | FDNKQDKKIAPETHRSKEVPLNERICLQVGSQ | |
| | CSTNESDNPSILVEKCTPPPEGDPESAVTELQC | |
| | VWHNLSYMKCTWLPGRNTSPDTNYTLYYWH | |
| | SSLGKILQCEDIYREGQHIGCSFALTNLKDSSFE | |
| | QHSVQIVVKDNAGKIRPSFNIVPLTSHVKPDPP | |
| | HIKRLFFQNGNLYVQWKNPQNFYSRCLSYQV | |
| | EVNNSQTETNDIFYVEEAKCQNSEFEGNLEGTI | |
| | CFMVPGVLPDTLNTVRIRVRTNKLCYEDDKL | |
| | WSNWSQAMSIGENTDPTPKRENGRVPRPPDCP | |
| | KCPAPEMLGGPSVFIFPPKPKDTLLIARTPEVT | |
| | CVVVDLDPEDPEVQISWFVDGKQMQTAKTQP | |
| | REEQFNGTYRVVSVLPIGHQDWLKGKQFTCK | |
| | VNNKALPSPIERTISKARGQAHQPSVYVLPPSR | |
| | EELSKNTVSLTCLIKDFFPPDIDVEWQSNGQQE | |
| | PESKYRTTPPQLDEDGSYFLYSKLSVDKSRWQ | |
| | RGDTFICAVMHEALHNHYTQESLSHSPGK | |
| 16 | SGSVKVLHEPSCFSDYISTSVCQWKMDHPTNC | Canis lupus |
| | SAELRLSYQLDFMGSENHTCVPENREDSVCVC | IL4R-IL13R-IgGC (without |
| | SMPIDDAVEADVYQLDLWAGQQLLWSGSFQP | signal sequence) |
| | SKHVKPRTPGNLTVHPNISHTWLLMWTNPYPT | |
| | ENHLHSELTYMVNVSNDNDPEDFKVYNVTY | |
| | MGPTLRLAASTLKSGASYSARVRAWAQTYNS | |
| | TWSDWSPSTTWLNYYEPGGGSGTETQPPVTN | |
| | LSVSVENLCTVIWTWDPPEGASPNCTLRYFSH | |
| | FDNKQDKKIAPETHRSKEVPLNERICLQVGSQ | |
| | CSTNESDNPSILVEKCTPPPEGDPESAVTELQC | |
| | VWHNLSYMKCTWLPGRNTSPDTNYTLYYWH | |
| | SSLGKILQCEDIYREGQHIGCSFALTNLKDSSFE | |
| | QHSVQIVVKDNAGKIRPSFNIVPLTSHVKPDPP | |
| | HIKRLFFQNGNLYVQWKNPQNFYSRCLSYQV | |
| | EVNNSQTETNDIFYVEEAKCQNSEFEGNLEGTI | |
| | CFMVPGVLPDTLNTVRIRVRTNKLCYEDDKL | |
| | WSNWSQAMSIGENTDPTAKECECKCNCNNCP | |
| | CPGCGLLGGPSVFIFPPKPKDILVTARTPTVTC | |
| | VVVDLDPENPEVQISWFVDSKQVQTANTQPRE | |
| | EQSNGTYRVVSVLPIGHQDWLSGKQFKCKVN | |
| | NKALPSPIEEIISKTPGQAHQPNVYVLPPSRDE | |
| | MSKNTVTLTCLVKDFFPPEIDVEWQSNGQQEP | |
| | ESKYRMTPPQLDEDGSYFLYSKLSVDKSRWQ | |
| | RGDTFICAVMHEALHNHYTQISLSHSPGK | |
| 17 | SGSVKVLHEPSCFSDYISTSVCQWKMDHPTNC | Canis lupus |
| | SAELRLSYQLDFMGSENHTCVPENREDSVCVC | IL4R-IL13R-IgGD |
| | SMPIDDAVEADVYQLDLWAGQQLLWSGSFQP | (without signal sequence) |
| | SKHVKPRTPGNLTVHPNISHTWLLMWTNPYPT | |
| | ENHLHSELTYMVNVSNDNDPEDFKVYNVTY | |
| | MGPTLRLAASTLKSGASYSARVRAWAQTYNS | |
| | TWSDWSPSTTWLNYYEPGGGSGTETQPPVTN | |
| | LSVSVENLCTVIWTWDPPEGASPNCTLRYFSH | |
| | FDNKQDKKIAPETHRSKEVPLNERICLQVGSQ | |
| | CSTNESDNPSILVEKCTPPPEGDPESAVTELQC | |
| | VWHNLSYMKCTWLPGRNTSPDTNYTLYYWH | |
| | SSLGKILQCEDIYREGQHIGCSFALTNLKDSSFE | |
| | QHSVQIVVKDNAGKIRPSFNIVPLTSHVKPDPP | |
| | HIKRLFFQNGNLYVQWKNPQNFYSRCLSYQV | |
| | EVNNSQTETNDIFYVEEAKCQNSEFEGNLEGTI | |
| | CFMVPGVLPDTLNTVRIRVRTNKLCYEDDKL | |
| | WSNWSQAMSIGENTDPTPKESTCKCISPCPVPE | |
| | SLGGPSVFIFPPKPKDILRITRTPEITCVVLDLGR | |
| | EDPEVQISWFVDGKEVHTAKTQPREQQFNSTY | |
| | RVVSVLPIEHQDWLTGKEFKCRVNHIGLPSPIE | |
| | RTISKARGQAHQPSVYVLPPSPKELSSSDTVTL | |
| | TCLIKDFFPPEIDVEWQSNGQPEPESKYHTTAP | |
| | QLDEDGSYFLYSKLSVDKSRWQQGDTFTCAV | |
| | MHEALQNHYTDLSLSHSPGK | |
| 18 | SGSVKVLRAPTCFSDYFSTSVCQWNMDAPTN | Felis catus |
| | CSAELRLSYQLNFMGSENRTCVPENGEGAAC | IL4R-IL13R (without signal |
| | ACSMLMDDFVEADVYQLHLWAGTQLLWSGS | sequence) |
| | FKPSSHVKPRAPGNLTVHPNVSHTWLLRWSN | |
| | PYPPENHLHAELTYMVNISSEDDPTDVSVCAS | |
| | GFLCHLLGLRRVETGAPGARLPPWLCAPRPRR | |
| | VPGSQCAVISCCRWVLIALTSRGGRWRLTPGL | |
| | RSQTRYVSVAEGLFGATPRVLCPGTQAGLASA | |
| | AREQMSPDPSAFHSIDYEPGGGSGSQTQPPVT | |
| | NLSVSVENLCTVIWTWDPPEGASPNCTLRYFS | |
| | HFDNKQDKKIAPETHRSKEVPLNERICLQVGS | |
| | QCSTNESDNPSILVEKCTPPPEGDPESAVTELQ | |
| | CVWHNLSYMKCTWLPGRNTSPDTNYTLYYW | |
| | HSSLGKILQCENIYREGQHIGCSFALTNLKDSS | |
| | FEQHSVQIVVKDNAGKIRPSFNIVPLTSHVKPD | |
| | PPHIKRLFFQNGNLYVQWKNPQNFYSRCLSYQ | |
| | VEVNNSQTETHDIFYVEEAKCQNSEFEGNLEG | |
| | TICFMVPGILPDTLNTVRIRVRTNKLCYEDDRL | |
| | WSNWSQAMSIGENTDPT | |
| 19 | SGSVKVLHLTACFSDYISASTCEWKMDRPTNC | Equus caballus |
| | SAQLRLSYQLNDEFSDNLTCIPENREDEVCVC | IL4R-IL13R (without signal |
| | RMLMDNIVSEDVYELDLWAGNQLLWNSSFKP | sequence) |
| | SRHVKPRAPQNLTVHAISHTWLLTWSNPYPLK | |
| | NHLWSELTYLVNISKEDDPTDFKIYNVTYMDP | |
| | TLRVTASTLKSRATYSARVKARAQNYNSTWS | |
| | EWSPSTTWHNYYEQPGGGSGTESQPPVTNLSV | |
| | SVENLCTVIWTWNPPEGVSPNCSLWYFSHFGN | |
| | KQDKKIAPETHRSKEVPLNERICLQVGSQCSTN | |
| | ESDNPSILVEKCISPPEGDPESAVTELQCVWHN | |
| | LSYMKCTWLPGKNASPDTNYTLYYWHSSLGK | |
| | ILQCEDIYREGQHIGCSFALTEVKDSIFEQHSVQ | |
| | IIVIVKDNAGKIRPFFNIVPLTSHVKPDPPHIKKL | |
| | FFQNGDLYVQWKNPQNFYSRCLSYQVEVNNS | |
| | QTETRDIFSVEEAKCQNPEFEGDLEGTICFMVP | |
| | GVLPDTVNTVRIRVKTNKLCYEDDKLWSNWS | |
| | QAMSIGKKADPT | |
| 20 | MAVLGLLFCLVTFPSCVLSTETQPPVTNLSVSV | Canis lupus |
| | ENLCTVIWTWDPPEGASPNCTLRYFSHFDNKQ | IL13R-IL4R-IgGB |
| | DKKIAPETHRSKEVPLNERICLQVGSQCSTNES | (with signal sequence) |
| | DNPSILVEKCTPPPEGDPESAVTELQCVWHNLS | |
| | YMKCTWLPGRNTSPDTNYTLYYWHSSLGKIL | |
| | QCEDIYREGQHIGCSFALTNLKDSSFEQHSVQI | |
| | VVKDNAGKIRPSFNIVPLTSHVKPDPPHIKRLF | |
| | FQNGNLYVQWKNPQNFYSRCLSYQVEVNNSQ | |
| | TETNDIFYVEEAKCQNSEFEGNLEGTICFMVPG | |
| | VLPDTLNTVRIRVRTNKLCYEDDKLWSNWSQ | |
| | AMSIGENTDPTGGGSGSGSVKVLHEPSCFSDYI | |
| | STSVCQWKMDHPTNCSAELRLSYQLDFMGSE | |
| | NHTCVPENREDSVCVCSMPIDDAVEADVYQL | |
| | DLWAGQQLLWSGSFQPSKHVKPRTPGNLTVH | |
| | PNISHTWLLMWTNPYPTENHLHSELTYMVNV | |
| | SNDNDPEDFKVYNVTYMGPTLRLAASTLKSG | |
| | ASYSARVRAWAQTYNSTWSDWSPSTTWLNY | |
| | YEPKRENGRVPRPPDCPKCPAPEMLGGPSVFIF | |
| | PPKPKDTLLIARTPEVTCVVVDLDPEDPEVQIS | |
| | WFVDGKQMQTAKTQPREEQFNGTYRVVSVLP | |
| | IGHQDWLKGKQFTCKVNNKALPSPIERTISKA | |
| | RGQAHQPSVYVLPPSREELSKNTVSLTCLIKDF | |
| | FPPDIDVEWQSNGQQEPESKYRTTPPQLDEDG | |
| | SYFLYSKLSVDKSRWQRGDTFICAVMHEALH | |
| | NHYTQESLSHSPGK | |
| 21 | MAVLGLLFCLVTFPSCVLSSGSVKVLHEPSCFS | Canis lupus |
| | DYISTSVCQWKMDHPTNCSAELRLSYQLDFM | IL4R-IL13R-IgGB |
| | GSENHTCVPENREDSVCVCSMPIDDAVEADV | (with signal sequence) |
| | YQLDLWAGQQLLWSGSFQPSKHVKPRTPGNL | |
| | TVHPNISHTWLLMWTNPYPTENHLHSELTYM | |
| | VNVSNDNDPEDFKVYNVTYMGPTLRLAASTL | |
| | KSGASYSARVRAWAQTYNSTWSDWSPSTTW | |
| | LNYYEPGGGSGTETQPPVTNLSVSVENLCTVI | |
| | WTWDPPEGASPNCTLRYFSHFDNKQDKKIAPE | |
| | THRSKEVPLNERICLQVGSQCSTNESDNPSILV | |
| | EKCTPPPEGDPESAVTELQCVWHNLSYMKCT | |
| | WLPGRNTSPDTNYTLYYWHSSLGKILQCEDIY | |
| | REGQHIGCSFALTNLKDSSFEQHSVQIVVKDN | |
| | AGKIRPSFNIVPLTSHVKPDPPHIKRLFFQNGNL | |
| | YVQWKNPQNFYSRCLSYQVEVNNSQTETNDI | |
| | FYVEEAKCQNSEFEGNLEGTICFMVPGVLPDT | |
| | LNTVRIRVRTNKLCYEDDKLWSNWSQAMSIG | |
| | ENTDPTPKRENGRVPRPPDCPKCPAPEMLGGP | |
| | SVFIFPPKPKDTLLIARTPEVTCVVVDLDPEDPE | |
| | VQISWFVDGKQMQTAKTQPREEQFNGTYRVV | |
| | SVLPIGHQDWLKGKQFTCKVNNKALPSPIERTI | |
| | SKARGQAHQPSVYVLPPSREELSKNTVSLTCLI | |
| | KDFFPPDIDVEWQSNGQQEPESKYRTTPPQLD | |
| | EDGSYFLYSKLSVDKSRWQRGDTFICAVMHE | |
| | ALHNHYTQESLSHSPGK | |
| 22 | TETQPPVTNLSVSVENLCTVIWTWDPPEGASP | Canis lupus IL13R |
| | NCTLRYFSHFDNKQDKKIAPETHRSKEVPLNE | extracellular domain |
| | RICLQVGSQCSTNESDNPSILVEKCTPPPEGDPE | (without signal sequence) |
| | SAVTELQCVWHNLSYMKCTWLPGRNTSPDTN | |
| | YTLYYWHSSLGKILQCEDIYREGQHIGCSFALT | |
| | NLKDSSFEQHSVQIVVKDNAGKIRPSFNIVPLT | |
| | SHVKPDPPHIKRLFFQNGNLYVQWKNPQNFYS | |
| | RCLSYQVEVNNSQTETNDIFYVEEAKCQNSEF | |
| | EGNLEGTICFMVPGVLPDTLNTVRIRVRTNKL | |
| | CYEDDKLWSNWSQAMSIGENTDPT | |
| 23 | SGSGSVKVLHEPSCFSDYISTSVCQWKMDHPT | Canis lupus IL4R |
| | NCSAELRLSYQLDFMGSENHTCVPENREDSVC | extracellular domain |
| | VCSMPIDDAVEADVYQLDLWAGQQLLWSGSF | (without signal sequence) |
| | QPSKHVKPRTPGNLTVHPNISHTWLLMWTNP | |
| | YPTENHLHSELTYMVNVSNDNDPEDFKVYNV | |
| | TYMGPTLRLAASTLKSGASYSARVRAWAQTY | |
| | NSTWSDWSPSTTWLNYYEP | |
| 24 | SQTQPPVTNLSVSVENLCTVIWTWDPPEGASP | Felis catus IL13R |
| | NCTLRYFSHFDNKQDKKIAPETHRSKEVPLNE | extracellular domain |
| | RICLQVGSQCSTNESDNPSILVEKCTPPPEGDPE | (without signal sequence) |
| | SAVTELQCVWHNLSYMKCTWLPGRNTSPDTN | |
| | YTLYYWHSSLGKILQCENIYREGQHIGCSFALT | |
| | NLKDSSFEQHSVQIVVKDNAGKIRPSFNIVPLT | |
| | SHVKPDPPHIKRLFFQNGNLYVQWKNPQNFYS | |
| | RCLSYQVEVNNSQTETHDIFYVEEAKCQNSEF | |
| | EGNLEGTICFMVPGILPDTLNTVRIRVRTNKLC | |
| | YEDDRLWSNWSQAMSIGENTDPT | |
| 25 | SGSVKVLRAPTCFSDYFSTSVCQWNMDAPTN | Felis catus IL4R |
| | CSAELRLSYQLNFMGSENRTCVPENGEGAAC | extracellular domain |
| | ACSMLMDDFVEADVYQLHLWAGTQLLWSGS | (without signal sequence) |
| | FKPSSHVKPRAPGNLTVHPNVSHTWLLRWSN | |
| | PYPPENHLHAELTYMVNISSEDDPTDVSVCAS | |
| | GFLCHLLGLRRVETGAPGARLPPWLCAPRPRR | |
| | VPGSQCAVISCCRWVLIALTSRGGRWRLTPGL | |
| | RSQTRYVSVAEGLFGATPRVLCPGTQAGLASA | |
| | AREQMSPDPSAFHSIDYEP | |
| 26 | TESQPPVTNLSVSVENLCTVIWTWNPPEGVSP | Equus caballus IL13R |
| | NCSLWYFSHFGNKQDKKIAPETHRSKEVPLNE | extracellular domain |
| | RICLQVGSQCSTNESDNPSILVEKCISPPEGDPE | (without signal sequence) |
| | SAVTELQCVWHNLSYMKCTWLPGKNASPDT | |
| | NYTLYYWHSSLGKILQCEDIYREGQHIGCSFA | |
| | LTEVKDSIFEQHSVQIMVKDNAGKIRPFFNIVP | |
| | LTSHVKPDPPHIKKLFFQNGDLYVQWKNPQNF | |
| | YSRCLSYQVEVNNSQTETRDIFSVEEAKCQNP | |
| | EFEGDLEGTICFMVPGVLPDTVNTVRIRVKTN | |
| | KLCYEDDKLWSNWSQAMSIGKKADPT | |
| 27 | SGSVKVLHLTACFSDYISASTCEWKMDRPTNC | Equus caballus IL4R |
| | SAQLRLSYQLNDEFSDNLTCIPENREDEVCVC | extracellular domain |
| | RMLMDNIVSEDVYELDLWAGNQLLWNSSFKP | (without signal sequence) |
| | SRHVKPRAPQNLTVHAISHTWLLTWSNPYPLK | |
| | NHLWSELTYLVNISKEDDPTDFKIYNVTYMDP | |
| | TLRVTASTLKSRATYSARVKARAQNYNSTWS | |
| | EWSPSTTWHNYYEQP | |
| 28 | SQTQPPVTNLSVSVENLCTVIWTWDPPEGASP | Feline |
| | NCTLRYFSHFDNKQDKKIAPETHRSKEVPLNE | IL13R-IL4R-IgG2 (without |
| | RICLQVGSQCSTNESDNPSILVEKCTPPPEGDPE | signal sequence) |
| | SAVTELQCVWHNLSYMKCTWLPGRNTSPDTN | |
| | YTLYYWHSSLGKILQCENIYREGQHIGCSFALT | |
| | NLKDSSFEQHSVQIVVKDNAGKIRPSFNIVPLT | |
| | SHVKPDPPHIKRLFFQNGNLYVQWKNPQNFYS | |
| | RCLSYQVEVNNSQTETHDIFYVEEAKCQNSEF | |
| | EGNLEGTICFMVPGILPDTLNTVRIRVRTNKLC | |
| | YEDDRLWSNWSQAMSIGENTDPTGGGSGSSG | |
| | SVKVLRAPTCFSDYFSTSVCQWNMDAPTNCS | |
| | AELRLSYQLNFMGSENRTCVPENGEGAACAC | |
| | SMLMDDFVEADVYQLHLWAGTQLLWSGSFK | |
| | PSSHVKPRAPGNLTVHPNVSHTWLLRWSNPY | |
| | PPENHLHAELTYMVNISSEDDPTDVSVCASGF | |
| | LCHLLGLRRVETGAPGARLPPWLCAPRPRRVP | |
| | GSQCAVISCCRWVLIALTSRGGRWRLTPGLRS | |
| | QTRYVSVAEGLFGATPRVLCPGTQAGLASAA | |
| | REQMSPDPSAFHSIDYEPSPKTASTIESKTGECP | |
| | KCPVPEIPGAPSVFIFPPKPKDTLSISRTPEVTCL | |
| | VVDLGPDDSNVQITWFVDNTEMHTAKTRPRE | |
| | EQFNSTYRVVSVLPILHQDWLKGKEFKCKVNS | |
| | KSLPSAMERTISKAKGQPHEPQVYVLPPTQEEL | |
| | SENKVSVTCLIKGFHPPDIAVEWEITGQPEPEN | |
| | NYQTTPPQLDSDGTYFLYSRLSVDRSHWQRG | |
| | NTYTCSVSHEALHSHHTQKSLTQSPGK | |
| 29 | SGSVKVLRAPTCFSDYFSTSVCQWNMDAPTN | Feline |
| | CSAELRLSYQLNFMGSENRTCVPENGEGAAC | IL4R-IL13R-IgG2 (without |
| | ACSMLMDDFVEADVYQLHLWAGTQLLWSGS | signal sequence) |
| | FKPSSHVKPRAPGNLTVHPNVSHTWLLRWSN | |
| | PYPPENHLHAELTYMVNISSEDDPTDVSVCAS | |
| | GFLCHLLGLRRVETGAPGARLPPWLCAPRPRR | |
| | VPGSQCAVISCCRWVLIALTSRGGRWRLTPGL | |
| | RSQTRYVSVAEGLFGATPRVLCPGTQAGLASA | |
| | AREQMSPDPSAFHSIDYEPGGGSGSSQTQPPVT | |
| | NLSVSVENLCTVIWTWDPPEGASPNCTLRYFS | |
| | HFDNKQDKKIAPETHRSKEVPLNERICLQVGS | |
| | QCSTNESDNPSILVEKCTPPPEGDPESAVTELQ | |
| | CVWHNLSYMKCTWLPGRNTSPDTNYTLYYW | |
| | HSSLGKILQCENIYREGQHIGCSFALTNLKDSS | |
| | FEQHSVQIVVKDNAGKIRPSFNIVPLTSHVKPD | |
| | PPHIKRLFFQNGNLYVQWKNPQNFYSRCLSYQ | |
| | VEVNNSQTETHDIFYVEEAKCQNSEFEGNLEG | |
| | TICFMVPGILPDTLNTVRIRVRTNKLCYEDDRL | |
| | WSNWSQAMSIGENTDPTSPKTASTIESKTGECP | |
| | KCPVPEIPGAPSVFIFPPKPKDTLSISRTPEVTCL | |
| | VVDLGPDDSNVQITWFVDNTEMHTAKTRPRE | |
| | EQFNSTYRVVSVLPILHQDWLKGKEFKCKVNS | |
| | KSLPSAMERTISKAKGQPHEPQVYVLPPTQEEL | |
| | SENKVSVTCLIKGFHPPDIAVEWEITGQPEPEN | |
| | NYQTTPPQLDSDGTYFLYSRLSVDRSHWQRG | |
| | NTYTCSVSHEALHSHHTQKSLTQSPGK | |
| 30 | TESQPPVTNLSVSVENLCTVIWTWNPPEGVSP | Equine |
| | NCSLWYFSHFGNKQDKKIAPETHRSKEVPLNE | IL13R-IL4R-IgG2 (without |
| | RICLQVGSQCSTNESDNPSILVEKCISPPEGDPE | signal sequence) |
| | SAVTELQCVWHNLSYMKCTWLPGKNASPDT | |
| | NYTLYYWHSSLGKILQCEDIYREGQHIGCSFA | |
| | LTEVKDSIFEQHSVQIMVKDNAGKIRPFFNIVP | |
| | LTSHVKPDPPHIKKLFFQNGDLYVQWKNPQNF | |
| | YSRCLSYQVEVNNSQTETRDIFSVEEAKCQNP | |
| | EFEGDLEGTICFMVPGVLPDTVNTVRIRVKTN | |
| | KLCYEDDKLWSNWSQAMSIGKKADPTGGGS | |
| | GSSGSVKVLHLTACFSDYISASTCEWKMDRPT | |
| | NCSAQLRLSYQLNDEFSDNLTCIPENREDEVC | |
| | VCRMLMDNIVSEDVYELDLWAGNQLLWNSSF | |
| | KPSRHVKPRAPQNLTVHAISHTWLLTWSNPYP | |
| | LKNHLWSELTYLVNISKEDDPTDFKIYNVTYM | |
| | DPTLRVTASTLKSRATYSARVKARAQNYNST | |
| | WSEWSPSTTWHNYYEQPDMSKCPKCPAPELL | |
| | GGPSVFIFPPNPKDTLMISRTPVVTCVVVNLSD | |
| | QYPDVQFSWYVDNTEVHSAITKQREAQFNST | |
| | YRVVSVLPIQHQDWLSGKEFKCSVTNVGVPQP | |
| | ISRAISRGKGPSRVPQVYVLPPHPDELAKSKVS | |
| | VTCLVKDFYPPDISVEWQSNRWPELEGKYSTT | |
| | PAQLDGDGSYFLYSKLSLETSRWQQVESFTCA | |
| | VMHEALHNHYTKTDISESLGK | |
| 31 | SGSVKVLHLTACFSDYISASTCEWKMDRPTNC | Equine |
| | SAQLRLSYQLNDEFSDNLTCIPENREDEVCVC | IL4R-IL13 R-IgG2 (without |
| | RMLMDNIVSEDVYELDLWAGNQLLWNSSFKP | signal sequence) |
| | SRHVKPRAPQNLTVHAISHTWLLTWSNPYPLK | |
| | NHLWSELTYLVNISKEDDPTDFKIYNVTYMDP | |
| | TLRVTASTLKSRATYSARVKARAQNYNSTWS | |
| | EWSPSTTWHNYYEQPGGGSGSTESQPPVTNLS | |
| | VSVENLCTVIWTWNPPEGVSPNCSLWYFSHFG | |
| | NKQDKKIAPETHRSKEVPLNERICLQVGSQCST | |
| | NESDNPSILVEKCISPPEGDPESAVTELQCVWH | |
| | NLSYMKCTWLPGKNASPDTNYTLYYWHSSLG | |
| | KILQCEDIYREGQHIGCSFALTEVKDSIFEQHSV | |
| | QIMVKDNAGKIRPFFNIVPLTSHVKPDPPHIKK | |
| | LFFQNGDLYVQWKNPQNFYSRCLSYQVEVNN | |
| | SQTETRDIFSVEEAKCQNPEFEGDLEGTICFMV | |
| | PGVLPDTVNTVRIRVKTNKLCYEDDKLWSNW | |
| | SQAMSIGKKADPTDMSKCPKCPAPELLGGPSV | |
| | FIFPPNPKDTLMISRTPVVTCVVVNLSDQYPDV | |
| | QFSWYVDNTEVHSAITKQREAQFNSTYRVVS | |
| | VLPIQHQDWLSGKEFKCSVTNVGVPQPISRAIS | |
| | RGKGPSRVPQVYVLPPHPDELAKSKVSVTCLV | |
| | KDFYPPDISVEWQSNRWPELEGKYSTTPAQLD | |
| | GDGSYFLYSKLSLETSRWQQVESFTCAVMHE | |
| | ALHNHYTKTDISESLGK | |
| 32 | QPPVTNLSVSVENLCTVIWTWDPPEGASPNCT | Canine mini-IL13R ECD |
| | LRYFSHFDNKQDKKIAPETHRSKEVPLNERICL | |
| | QVGSQCSTNESDNPSILVEKCTPPPEGDPESAV | |
| | TELQCVWHNLSYMKCTWLPGRNTSPDTNYTL | |
| | YYWHSSLGKILQCEDIYREGQHIGCSFALTNL | |
| | KDSSFEQHSVQIVVKDNAGKIRPSFNIVPLTSH | |
| | VKPDPPHIKRLFFQNGNLYVQWKNPQNFYSR | |
| | CLSYQVEVNNSQTETNDIFYVEEAKCQNSEFE | |
| | GNLEGTICFMVPGVLPDTLNTVRIRVRTNKLC | |
| | YEDDKLWSNWSQAMSI | |
| 33 | KVLHEPSCFSDYISTSVCQWKMDHPTNCSAEL | Canine mini-IL4R ECD |
| | RLSYQLDFMGSENHTCVPENREDSVCVCSMPI | |
| | DDAVEADVYQLDLWAGQQLLWSGSFQPSKH | |
| | VKPRTPGNLTVHPNISHTWLLMWTNPYPTEN | |
| | HLHSELTYMVNVSNDNDPEDFKVYNVTYMGP | |
| | TLRLAASTLKSGASYSARVRAWAQTYNS | |
| 34 | QPPVTNLSVSVENLCTVIWTWDPPEGASPNCT | Feline mini-IL13R ECD |
| | LRYFSHFDNKQDKKIAPETHRSKEVPLNERICL | |
| | QVGSQCSTNESDNPSILVEKCTPPPEGDPESAV | |
| | TELQCVWHNLSYMKCTWLPGRNTSPDTNYTL | |
| | YYWHSSLGKILQCENIYREGQHIGCSFALTNL | |
| | KDSSFEQHSVQIVVKDNAGKIRPSFNIVPLTSH | |
| | VKPDPPHIKRLFFQNGNLYVQWKNPQNFYSR | |
| | CLSYQVEVNNSQTETHDIFYVEEAKCQNSEFE | |
| | GNLEGTICFMVPGILPDTLNTVRIRVRTNKLCY | |
| | EDDRLWSNWSQAMSI | |
| 35 | KVLRAPTCFSDYFSTSVCQWNMDAPTNCSAE | Feline mini-IL4R ECD |
| | LRLSYQLNFMGSENRTCVPENGEGAACACSM | |
| | LMDDFVEADVYQLHLWAGTQLLWSGSFKPSS | |
| | HVKPRAPGNLTVHPNVSHTWLLRWSNPYPPE | |
| | NHLHAELTYMVNISSEDDPTDVSVCASGFLCH | |
| | LLGLRRVETGAPGARLPPWLCAPRPRRVPGSQ | |
| | CAVISCCRWVLIALTSRGGRWRLTPGLRSQTR | |
| | YVSVAEGLFGATPRVLCPGTQAGLASAAREQ | |
| | MSPDPSAFHSIDYEP | |
| 36 | QPPVTNLSVSVENLCTVIWTWNPPEGVSPNCS | Equine mini-IL13R ECD |
| | LWYFSHFGNKQDKKIAPETHRSKEVPLNERIC | |
| | LQVGSQCSTNESDNPSILVEKCISPPEGDPESA | |
| | VTELQCVWHNLSYMKCTWLPGKNASPDTNY | |
| | TLYYWHSSLGKILQCEDIYREGQHIGCSFALTE | |
| | VKDSIFEQHSVQIMVKDNAGKIRPFFNIVPLTS | |
| | HVKPDPPHIKKLFFQNGDLYVQWKNPQNFYS | |
| | RCLSYQVEVNNSQTETRDIFSVEEAKCQNPEFE | |
| | GDLEGTICFMVPGVLPDTVNTVRIRVKTNKLC | |
| | YEDDKLWSNWSQAMSI | |
| 37 | KVLHLTACFSDYISASTCEWKMDRPTNCSAQL | Equine mini-IL4R ECD |
| | RLSYQLNDEFSDNLTCIPENREDEVCVCRMLM | |
| | DNIVSEDVYELDLWAGNQLLWNSSFKPSRHV | |
| | KPRAPQNLTVHAISHTWLLTWSNPYPLKNHL | |
| | WSELTYLVNISKEDDPTDFKIYNVTYMDPTLR | |
| | VTASTLKSRATYSARVKARAQNYNSTWSEWS | |
| | PSTTWHNYYEQP | |

### DESCRIPTION OF THE EMBODIMENTS

Contiguous polypeptides that bind canine IL13 and/or IL4, feline IL13 and/or IL4, and/or equine IL13 and/or IL4 are provided. In some embodiments, the contiguous polypeptides comprise an extracellular domain of an IL13R polypeptide and an extracellular domain of an IL4R polypeptide. Methods of producing or purifying contiguous polypeptides are also provided. Methods of treatment using contiguous polypeptides to bind IL13 and/or IL4 and inhibit IL13- and/or IL-4-mediated signaling are provided. Such methods include, but are not limited to, methods of treating IL13- and/or IL4-induced conditions in companion animal species. Methods of detecting IL13 and/or IL4 in a sample from a companion animal species are also provided.

For the convenience of the reader, the following definitions of terms used herein are provided.

As used herein, numerical terms such as Kd are calculated based upon scientific measurements and, thus, are subject to appropriate measurement error. In some instances, a numerical term may include numerical values that are rounded to the nearest significant figure.

As used herein, “a” or “an” means “at least one” or “one or more” unless otherwise specified. As used herein, the term “or” means “and/or” unless specified otherwise. In the context of a multiple dependent claim, the use of “or” when referring back to other claims refers to those claims in the alternative only.

### IL13R/IL4R Contiguous Polypeptides

Novel IL13R/IL4R contiguous polypeptides are provided, for example, contiguous polypeptides that bind canine IL13 and/or IL4, feline IL13 and/or IL4, and/or equine IL13 and/or IL4.

“Amino acid sequence,” means a sequence of amino acids residues in a peptide or protein. The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

The term “contiguous polypeptide” herein is used to mean an uninterrupted sequence of amino acids. A contiguous polypeptide is typically translated from a single continuous DNA sequence. It can be made by genetic engineering, for example, by removing the stop codon from the DNA sequence of the first protein, then appending the DNA sequence of the second protein in frame, so that the DNA sequence is expressed as a single protein. Typically, this is accomplished by cloning a cDNA into an expression vector in frame with an existing gene.

“IL4R,” as used herein, is a polypeptide comprising the entirety or a fragment of IL4 receptor subunit alpha that bind to IL-4.

For example, “IL4R” refers to an IL4R polypeptide from any vertebrate source, including mammals such as primates (e.g., humans and cynomolgus monkeys), rodents (e.g., mice and rats), and companion animals (e.g., dogs, cats, and equine), unless otherwise indicated. In some embodiments, IL4R is an extracellular domain fragment that binds IL4. In some such embodiments, the IL4R may be referred to as an IL4R extracellular domain (ECD). In some embodiments, IL4R comprises the amino acid sequence of SEQ ID NO: 7, SEQ ID NO:8, SEQ ID NO: 9, SEQ ID NO: 23, SEQ ID NO: 25, SEQ ID NO: 27, SEQ ID NO: 33, SEQ ID NO: 35, or SEQ ID NO: 37.

“IL13R,” as used herein, is a polypeptide comprising the entirety or a portion of IL13 receptor subunit alpha-1 that binds to IL-13.

For example, “IL13R” refers to an IL13R polypeptide from any vertebrate source, including mammals such as primates (e.g., humans and cynomolgus monkeys), rodents (e.g., mice and rats), and companion animals (e.g., dogs, cats, and equine), unless otherwise indicated. In some embodiments, IL13R is an extracellular domain fragment that binds IL13. In some such embodiments, the IL13R may be referred to as an IL13R extracellular domain (ECD). In some embodiments, the IL13R polypeptide comprises the amino acid sequence of SEQ ID NO: 10, SEQ ID NO: 11, SEQ ID NO: 12, SEQ ID NO: 22, SEQ ID NO: 24, SEQ ID NO: 26, SEQ ID NO: 32, SEQ ID NO: 34, or SEQ ID NO: 36.

The term “companion animal species” refers to an animal suitable to be a companion to humans. In some embodiments, a companion animal species is a small mammal, such as a canine, feline, dog, cat, horse, rabbit, ferret, guinea pig, rodent, etc. In some embodiments, a companion animal species is a farm animal, such as a horse, cow, pig, etc.

An “extracellular domain” (“ECD”) is the portion of a polypeptide that extends beyond the transmembrane domain into the extracellular space. The term “extracellular domain,” as used herein, may comprise a complete extracellular domain or may comprise a truncated extracellular domain missing one or more amino acids, that binds to its ligand. The composition of the extracellular domain may depend on the algorithm used to determine which amino acids are in the membrane. Different algorithms may predict, and different systems may express, different extracellular domains for a given protein.

An extracellular domain of an IL4R polypeptide may comprise a complete extracellular domain or a truncated extracellular domain of IL4R that binds IL4. As used herein, the terms “extracellular domain of an IL4R polypeptide,” “IL4R ECD,” and similar terms refer to an IL4R polypeptide that does not comprise a transmembrane domain or cytoplasmic domain, even if the term follows an open transitional word, such as “comprising,” “comprises,” and the like. In some embodiments, an extracellular domain of an IL4R polypeptide is an extracellular domain of an IL4R polypeptide derived from a companion species animal. For example, in some embodiments, an extracellular domain of an IL4R polypeptide is derived from canine IL4R, feline IL4R or equine IL4R. In some embodiments, an extracellular domain of an IL4R polypeptide comprises the amino acid sequence of SEQ ID NO: 23, SEQ ID NO: 25, or SEQ ID NO: 27, or any fragment thereof. In some embodiments, an extracellular domain of an IL4R polypeptide comprises the amino acid sequence of SEQ ID NO: 33, SEQ ID NO: 35, or SEQ ID NO: 37, or any fragment thereof.

An extracellular domain of an IL13R polypeptide may comprise a complete extracellular domain or a truncated extracellular domain of IL13R that binds IL13. As used herein, the terms “extracellular domain of an IL13R polypeptide,” “IL13R ECD,” and similar terms refer to an IL13R polypeptide that does not comprise a transmembrane domain or cytoplasmic domain, even if the term follows an open transitional word, such as “comprising,” “comprises,” and the like. In some embodiments, an extracellular domain of an IL13R polypeptide is an extracellular domain of an IL13R polypeptide derived from a companion species animal. For example, in some embodiments, an extracellular domain of an IL13R polypeptide is derived from canine IL13R, feline IL13R or equine IL13R. In some embodiments, an extracellular domain of an IL13R polypeptide comprises the amino acid sequence of SEQ ID NO: 22, SEQ ID NO: 24, or SEQ ID NO: 26, or any fragment thereof. In some embodiments, an extracellular domain of an IL13R polypeptide comprises the amino acid sequence of SEQ ID NO: 32, SEQ ID NO: 34, or SEQ ID NO: 36, or any fragment thereof.

The terms “IL13R/IL4R contiguous polypeptide” and “IL4R/IL13R contiguous polypeptide” are used interchangeably to refer to a contiguous polypeptide comprising an IL13R polypeptide and an IL4R polypeptide, where the terms are not indicative of the order in which the IL13R and IL4R polypeptides appear in the contiguous polypeptide, unless the order is otherwise indicated. For example, an IL13R/IL4R contiguous polypeptide or an IL4R/IL13R contiguous polypeptide may refer to an IL4R polypeptide preceded in sequence or followed in sequence by an IL13R polypeptide. In addition, an IL13R/IL4R contiguous polypeptide or an IL4R/IL13R contiguous polypeptide may refer to an IL13R polypeptide preceded in sequence or followed in sequence by an IL4R polypeptide.

In some embodiments, the IL13R/IL4R contiguous polypeptide comprises an IL13R polypeptide joined to a IL4R polypeptide at the C-terminus of the IL13R polypeptide or at the N-terminus of the IL13R polypeptide. In some embodiments, the IL13R/IL4R contiguous polypeptide comprises an IL4R polypeptide joined to a IL13R polypeptide at the C-terminus of the IL4R polypeptide or at the N-terminus of the IL4R polypeptide.

The IL13R/IL4R contiguous polypeptide of the invention may comprise an extracellular domain of a IL13R polypeptide and/or an extracellular domain of a IL4R polypeptide, wherein the polypeptides are derived from a companion animal species. For example, a contiguous polypeptide may comprise an extracellular domain of an IL4R polypeptide from a dog, cat, or horse and/or may comprise an extracellular domain of an IL13R polypeptide from a dog, cat, or horse.

“Wild-type” refers to a non-mutated version of a polypeptide that occurs in nature, or a fragment thereof. A wild-type polypeptide may be produced recombinantly. A “wildtype IL13R ECD” or a “wildtype IL4R ECD” refers to a protein having an amino acid sequence that is identical to the same portion of an extracellular domain of an IL13R or IL4R that occurs in nature.

A “variant” is a nucleic acid molecule or polypeptide that differs from a referent nucleic acid molecule or polypeptide by single or multiple amino acid substitutions, deletions, and/or additions and substantially retains at least one biological activity of the referent nucleic acid molecule or polypeptide.

A “biologically active” entity, or an entity having “biological activity,” is an entity having any function related to or associated with a metabolic or physiological process, and/or having structural, regulatory, or biochemical functions of a naturally-occurring molecule. Biologically active polynucleotide fragments are those exhibiting similar activity, but not necessarily identical, to an activity of a polynucleotide of the present invention. A biologically active polypeptide or fragment thereof includes one that can participate in a biological reaction, including, but not limited to, a ligand-receptor interaction or antigen-antibody binding. The biological activity can include an improved desired activity, or a decreased undesirable activity. An entity may demonstrate biological activity when it participates in a molecular interaction with another molecule, such as hybridization, when it has therapeutic value in alleviating a disease condition, when it has prophylactic value in inducing an immune response, when it has diagnostic and/or prognostic value in determining the presence of a molecule, such as a biologically active fragment of a polynucleotide that may be detected as unique for the polynucleotide molecule, and when it can be used as a primer in a polymerase chain reaction (PCR).

As used herein, “percent (%) amino acid sequence identity” and “homology” with respect to a nucleic acid molecule or polypeptide sequence are defined as the percentage of nucleotide or amino acid residues in a referent sequence that are identical with the nucleotide or amino acid residues in the specific nucleic acid molecule or polypeptide sequence, after aligning the sequences and introducing gaps, if necessary to achieve the maximum percent sequence identity, and not considering any conservative substitutions as part of the sequence identity. Alignment for purposes of determining percent sequence identity can be achieved in various ways that are within the skill in the art, for instance, using publicly available computer software such as BLAST, BLAST-2, ALIGN, or MEGALINE™ (DNASTAR) software. Those skilled in the art can determine appropriate parameters for measuring alignment, including any algorithms needed to achieve maximal alignment over the full length of sequences being compared.

In some embodiments, a variant has at least about 50% sequence identity with the referent nucleic acid molecule or polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, deleted, at the N- or C-terminus of the polypeptide. In some embodiments, a variant has at least about 50% sequence identity, at least about 60% sequence identity, at least about 65% sequence identity, at least about 70% sequence identity, at least about 75% sequence identity, at least about 80% sequence identity, at least about 85% sequence identity, at least about 90% sequence identity, at least about 95% sequence identity, at least about 98% sequence identity with the sequence of the referent nucleic acid or polypeptide.

In some embodiments, an IL13R/IL4R contiguous polypeptide comprises an extracellular domain of an IL13R polypeptide having at least 85%, at least 90%, at least 95%, at least 98% sequence identity to the amino acid sequence of SEQ ID NO: 22, SEQ ID NO: 24, SEQ ID NO: 26, SEQ ID NO: 32, SEQ ID NO: 34, or SEQ ID NO: 36. In some embodiments, an IL13R/IL4R contiguous polypeptide comprises an extracellular domain of an IL4R polypeptide having at least 85%, at least 90%, at least 95%, at least 98% sequence identity to the amino acid sequence of SEQ ID NO: 23, SEQ ID NO: 25, SEQ ID NO: 27, SEQ ID NO: 33, SEQ ID NO: 35, or SEQ ID NO: 37.

In some embodiments, the IL13R/IL4R contiguous polypeptide comprises an extracellular domain of an IL13R polypeptide comprising a cysteine at a position corresponding to position 18 of SEQ ID NO: 22, at a position corresponding to position 18 of SEQ ID NO: 24, or at a position corresponding to position 18 of SEQ ID NO: 26. In some embodiments, the IL13R/IL4R contiguous polypeptide comprises an extracellular domain of an IL13R polypeptide comprising a cysteine at position 18 of SEQ ID NO: 22, at position 18 of SEQ ID NO: 24, at position 18 of SEQ ID NO: 26, at position 15 of SEQ ID NO: 32, at position 15 of SEQ ID NO: 34, or at position 15 of SEQ ID NO: 36.

A “point mutation” is a mutation that involves a single nucleotide or amino acid residue. The mutation may be the loss of a nucleotide or amino acid, substitution of one nucleotide or amino acid residue for another, or the insertion of an additional nucleotide or amino acid residue.

An amino acid substitution may include but is not limited to the replacement of one amino acid in a polypeptide with another amino acid. Exemplary substitutions are shown in Table 2. Amino acid substitutions may be introduced into a molecule of interest and the products screened for a desired activity, for example, retained/improved antigen binding, decreased immunogenicity, or improved ADCC or CDC or enhanced pharmacokinetics.

TABLE 2

| Original | |
|---|---|
| Residue | Exemplary Substitutions |
| Ala (A) | Val; Leu; Ile |
| Arg (R) | Lys; Gln; Asn |
| Asn (N) | Gln; His; Asp; Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; |
| | Norleucine |
| Leu (L) | Norleucine; Ile; Val; |
| | Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; |
| | Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes with another class.

A “fusion partner,” as used herein, refers to an additional component of an IL13R/IL4R contiguous polypeptide, such as an additional polypeptide, such as albumin, an albumin binding fragment, or a fragment of an immunoglobulin molecule. A fusion partner may comprise an oligomerization domain such as an Fec domain of a heavy chain immunoglobulin.

A “fragment crystallizable (Fec) polypeptide” is the portion of an antibody molecule that interacts with effector molecules and cells. It comprises the C-terminal portions of the immunoglobulin heavy chains. As used herein, an Fc polypeptide comprises a fragment of the Fc domain with one or more biological activity of an entire Fc polypeptide. An “effector function” of the Fc polypeptide is an action or activity performed in whole or in part by any antibody in response to a stimulus and may include complement fixation or ADCC (antibody-dependent cellular cytotoxicity) induction.

The term “IgX” or “IgX Fc” means the Fc region is derived from a particular antibody isotype (e.g., IgG, IgA, IgD, IgE, IgM, etc.), where “X” denotes the antibody isotype. Thus, “IgG” or “IgG Fc” denotes the Fc region of a γ chain, “IgA” or “IgA Fc” denotes the Fc region of an a chain, “IgD” or “IgD Fc” denotes the Fc region of a δ chain, “IgE” or “IgE Fc” denotes the Fc region of an c chain, “IgM” or “IgM Fc” denotes the Fc region of a chain, etc. In some embodiments, the IgG Fc region comprises CH1, hinge, CH2, CH3, and CL1. “IgXN” or “IgXN Fc” denotes that the Fc region is derived from a particular subclass of antibody isotype (such as canine IgG subclass A, B, C, or D; feline IgG subclass 1, 2a, or 2b; or equine IgG subclass IgG1, IgG2, IgG3, IgG4, IgG5, IgG6, or IgG7, etc.), where “N” denotes the subclass.

In some embodiments, IgX or IgXN regions are derived from a companion animal, such as a dog, a cat, or a horse. In some embodiments, IgG regions are isolated from canine γ heavy chains, such as IgGA, IgGB, IgGC, or IgGD. In some instances, IgG Fc regions are isolated from feline γ heavy chains, such as IgG1, IgG2a, or IgG2b. In other instances, IgG regions are isolated from equine γ heavy chains, such as IgG1, IgG2, IgG3, IgG4, IgG5, IgG6, or IgG7. Polypeptides comprising an Fc region of IgGA, IgGB, IgGC, or IgGD may provide for higher expression levels in recombination production systems.

A “signal sequence” refers to a sequence of amino acid residues or polynucleotides encoding such, which facilitates secretion of a polypeptide of interest and is typically cleaved upon export of the polypeptide to the outside of the cell surface membrane.

A “linker” refers to one or more amino acid residues that connects a first polypeptide with a second polypeptide.

In some embodiments, the linker is a glycine-rich, serine-rich, or GS-rich flexible, non-structural linker. In some embodiments, a linker comprises the amino acids G (Gly) and/or S (Ser). For example, a linker may comprise G or a repeat of G (e.g., GG, GGG, etc.); GS or a repeat of GS (e.g., GSGS (SEQ ID NO: 38), GSGSGS (SEQ ID NO: 39), etc.); GGS or a repeat of GGS (e.g., GGSGGS (SEQ ID NO: 40), GGSGGSGGS (SEQ ID NO: 41), etc.); GGGS (SEQ ID NO: 42) or a repeat of GGGS (SEQ ID NO: 42) (e.g., GGGSGGGS (SEQ ID NO: 43), GGGSGGGSGGGS (SEQ ID NO: 44), etc.); GSS or a repeat of GSS (e.g., GSSGSS (SEQ ID NO: 45), GSSGSSGSS (SEQ ID NO: 46), etc.); or GGSS (SEQ ID NO: 47) or a repeat of GGSS (SEQ ID NO: 47) (e.g., GGSSGGSS (SEQ ID NO: 48), GGSSGGSSGGSS (SEQ ID NO: 49), etc.).

In some embodiments, the IL13R/IL4R contiguous polypeptide comprises at least one fusion partner and/or at least one linker. In some embodiments, the IL13R/IL4R contiguous polypeptide comprises an optional signal sequence, an optional fusion partner, and at least one optional linker. In some embodiments, an IL13R/IL4R contiguous polypeptide does not comprise a signal sequence, a fusion partner, or a linker. In some embodiments, the IL13/IL4 contiguous polypeptide is translated with a signal sequence, but the signal sequence is cleaved from the IL13R/IL4R contiguous polypeptide.

In some embodiments, an IL13R/IL4R contiguous polypeptide comprises:

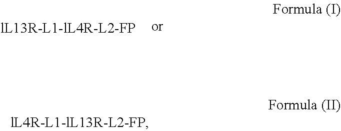

wherein IL13R is an IL13R extracellular domain (ECD) polypeptide derived from a companion animal species, IL4R is an IL4R ECD polypeptide derived from a companion animal species, L1 is a first optional linker, L2 is a second optional linker, and FP is an optional fusion partner.

In some embodiments, an IL13R/IL4R contiguous polypeptide comprises an amino acid sequence selected from SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 28, SEQ ID NO: 29, SEQ ID NO: 30, SEQ ID NO: 31.

### IL13R/IL4R Contiguous Polypeptide Expression and Production

Polynucleotide sequences that encode all or part (e.g., the extracellular domain) of an IL13R/IL4R contiguous polypeptide with or without a signal sequence are provided. If a homologous signal sequence (i.e., a signal sequence of native IL-4R or IL13R) is not used in the construction of the nucleic acid molecule, then another signal sequence may be used, for example, any one of the signal sequences described in PCT US06/02951.

Typically, nucleotide sequence encoding the polypeptide of interest, such as an IL13R/IL4R contiguous polypeptide, is inserted into an expression vector, suitable for expression in a selected host cell.

A “vector” is a plasmid that can be used to transfer DNA sequences from one organism to another or to express a gene of interest. A vector typically includes an origin of replication and regulatory sequences which regulate the expression of the gene of interest, and may or may not carry a selective marker gene, such as an antibiotic resistance gene. A vector is suitable for the host cell in which it is to be expressed. A vector may be termed a “recombinant vector” when the gene of interest is present in the vector.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NSO cells, PER.C6® cells (Crucell), 293 cells, and CHO cells, and their derivatives, such as 293-6E, DG44, CHO-S, and CHO-K cells. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) encoding an amino acid sequence(s) provided herein.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated.”

In some embodiments, the IL13R/IL4R contiguous polypeptide is isolated using chromatography, such as size exclusion chromatography, ion exchange chromatography, protein A column chromatography, hydrophobic interaction chromatography, and CHT chromatography.

The terms “label” and “detectable label” mean a moiety attached to a IL13R/IL4R contiguous polypeptide to render it detectable. In some embodiments, the label is a detectable marker that can produce a signal that is detectable by visual or instrumental means, for example, incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Examples of labels for polypeptides include, but are not limited to, the following: radioisotopes or radionuclides (for example, ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm); chromogens, fluorescent labels (for example, FITC, rhodamine, lanthanide phosphors), enzymatic labels (for example, horseradish peroxidase, luciferase, alkaline phosphatase); chemiluminescent markers; biotinyl groups; predetermined polypeptide epitopes recognized by a secondary reporter (for example, leucine zipper pair sequences, binding sites for secondary antibodies, metal binding domains, epitope tags); and magnetic agents, such as gadolinium chelates. Representative examples of labels commonly employed for immunoassays include moieties that produce light, for example, acridinium compounds, and moieties that produce fluorescence, for example, fluorescein. In this regard, the moiety itself may not be detectably labeled but may become detectable upon reaction with yet another moiety.

### IL13R/IL4R Contiguous Polypeptides as Decoy Receptor Traps

The IL13R/IL4R contiguous polypeptides of the invention can function as decoy receptors for trapping IL13 or IL4 and inhibiting their interaction with IL13R and IL4R on cell surfaces. Decoy receptors, such as those of the invention, recognize their ligands with high affinity and specificity but are structurally incapable of signaling. They compete with wild-type receptors for ligand binding and participate in ligand/receptor interactions, thus modulating the activity of or the number of functioning receptors and/or the cellular activity downstream from the receptors. Decoy receptors can act as molecular traps for agonist ligands and thereby inhibit ligand-induced receptor activation.

“IL13” as used herein refers to any native IL13 that results from expression and processing of IL13 in a cell. The term includes IL13 from any vertebrate source, including mammals such as primates (e.g., humans and cynomolgus monkeys) and rodents (e.g., mice and rats), and companion animals (e.g., dogs, cats, and equine), unless otherwise indicated. The term also includes naturally occurring variants of IL13, e.g., splice variants or allelic variants.

In some embodiments, a canine IL13 comprises the amino acid sequence of SEQ ID NO: 4. In some embodiments, a feline IL13 comprises the amino acid sequence of SEQ ID NO: 5. In some embodiments, an equine IL13 comprises the amino acid sequence of SEQ ID NO: 6.

“IL4” as used herein refers to any native IL4 that results from expression and processing of IL4 in a cell. The term includes IL4 from any vertebrate source, including mammals such as primates (e.g., humans and cynomolgus monkeys) and rodents (e.g., mice and rats), and companion animals (e.g., dogs, cats, and equine), unless otherwise indicated. The term also includes naturally occurring variants of IL4, e.g., splice variants or allelic variants.

In some embodiments, a canine IL4 comprises the amino acid sequence of SEQ ID NO: 1. In some embodiments, a feline IL4 comprises the amino acid sequence of SEQ ID NO: 2. In some embodiments, an equine IL4 comprises the amino acid sequence of SEQ ID NO: 3.

The invention provides IL13R/IL4R contiguous polypeptides as therapeutic agents. The IL13R/IL4R contiguous polypeptides of the invention bind to IL13 and/or IL4, described in more detail herein, which have been demonstrated to be associated with allergic diseases. In various embodiments, IL13R/IL4R contiguous polypeptides can bind IL13 and IL4 with very high affinity. In various embodiments, the IL13R/IL4R contiguous polypeptides can interfere with IL13 and IL4 signaling.

The term “affinity” means the strength of the sum total of noncovalent interactions between a single binding site of a molecule (for example, a receptor) and its binding partner (for example, a ligand). The affinity of a molecule X for its partner Y can generally be represented by the dissociation constant (K_{D}). Affinity can be measured by common methods known in the art, such as, for example, immunoblot, ELISA KD, KinEx A, biolayer interferometry (BLI), or surface plasmon resonance devices.

The terms “K_{D},” “K_{d},” “Kd” or “Kd value” as used interchangeably to refer to the equilibrium dissociation constant of a receptor fusion-ligand interaction. In some embodiments, the K_{d} of the fusion molecule to its ligand is measured by using biolayer interferometry assays using a biosensor, such as an Octet® System (Pall ForteBio LLC, Fremont, Calif.) according to the supplier's instructions. Briefly, biotinylated antigen is bound to the sensor tip and the association of fusion molecule is monitored for ninety seconds and the dissociation is monitored for 600 seconds. The buffer for dilutions and binding steps is 20 mM phosphate, 150 mM NaCl, pH 7.2. A buffer only blank curve is subtracted to correct for any drift. The data are fit to a 2:1 binding model using ForteBio data analysis software to determine association rate constant (kₒₙ), dissociation rate constant (k_{off}), and the K_{d}. The equilibrium dissociation constant (K_{d}) is calculated as the ratio of k_{off}/kₒₙ. The term “kon” refers to the rate constant for association of a molecule X to its partner Y and the term “koff” refers to the rate constant for dissociation of a molecule X or partner Y from the molecule X/partner Y complex.

The term “binds” to a substance is a term that is well understood in the art, and methods to determine such binding are also well known in the art. A molecule is said to exhibit “binding” if it reacts, associates with, or has affinity for a particular cell or substance and the reaction, association, or affinity is detectable by one or more methods known in the art, such as, for example, immunoblot, ELISA KD, KinEx A, biolayer interferometry (BLI), surface plasmon resonance devices, or etc.

“Surface plasmon resonance” denotes an optical phenomenon that allows for the analysis of real-time biospecific interactions by detection of alterations in protein concentrations within a biosensor matrix, for example using the BIAcore™ system (BIAcore International AB, a GE Healthcare company, Uppsala, Sweden and Piscataway, N.J.). For further descriptions, see Jonsson et al. (1993) Ann. Biol. Clin. 51: 19-26.

“Biolayer interferometry” refers to an optical analytical technique that analyzes the interference pattern of light reflected from a layer of immobilized protein on a biosensor tip and an internal reference layer. Changes in the number of molecules bound to the biosensor tip cause shifts in the interference pattern that can be measured in real-time. A nonlimiting exemplary device for biolayer interferometry is an Octet® system (Pall ForteBio LLC). See, e.g., Abdiche et al., 2008, Anal. Biochem. 377: 209-277.

In some embodiments, an IL13R/IL4R contiguous polypeptide binds to canine IL13 and/or IL4, feline IL13 and/or IL4, or equine IL13 and/or IL4 with a dissociation constant (Kd) of less than 5×10⁻⁶ M, less than 1×10⁻⁶ M, less than 5×10⁻⁷ M, less than 1×10⁻⁷ M, less than 5×10⁻⁸ M, less than 1×10⁻⁸ M, less than 5×10⁻⁹ M, less than 1×10⁻⁹ M, less than 5×10⁻¹⁰ M, less than 1×10⁻¹⁰ M, less than 5×10⁻¹¹ M, less than 1×10⁻¹¹ M, less than 5×10⁻¹² M, or less than 1×10⁻¹² M, as measured by biolayer interferometry. In some embodiments, an IL13R/IL4R contiguous polypeptide binds to canine IL13 and/or IL4, feline IL13 and/or IL4, or equine IL13 and/or IL4 with a Kd of between 5×10⁻⁶ M and 1×10⁻⁶ M, between 5×10⁻⁶ M and 5×10⁻⁷ M, between 5×10⁻⁶ M and 1×10⁻⁷ M, between 5×10⁻⁶ M and 5×10⁻⁸ M, 5×10⁻⁶ M and 1×10⁻⁸ M, between 5×10⁻⁶ M and 5×10⁻⁹ M, between 5×10⁻⁶ M and 1×10⁻⁹ M, between 5×10⁻⁶ M and 5×10⁻¹⁰ M, between 5×10⁻⁶ M and 1×10⁻¹⁰ M, between 5×10⁻⁶ M and 5×10⁻¹¹ M, between 5×10⁻⁶ M and 1×10⁻¹¹ M, between 5×10⁻⁶ M and 5×10⁻¹² M, between 5×10⁻⁶M and 1×10⁻¹² M, between 1×10⁻⁶ M and 5×10⁻⁷ M, between 1×10⁻⁶ M and 1×10⁻⁷ M, between 1×10⁻⁶ M and 5×10⁻⁸ M, 1×10⁻⁶ M and 1×10⁻⁸ M, between 1×10⁻⁶ M and 5×10⁻⁹M, between 1×10⁻⁶ M and 1×10⁻⁹ M, between 1×10⁻⁶ M and 5×10⁻¹⁰ M, between 1×10⁻⁶ M and 1×10⁻¹⁰ M, between 1×10⁻⁶ M and 5×10⁻¹¹ M, between 1×10⁻⁶ M and 1×10⁻¹¹ M, between 1×10⁻⁶ M and 5×10⁻¹² M, between 1×10⁻⁶ M and 1×10⁻¹² M, between 5×10⁻⁷ M and 1×10⁻⁷M, between 5×10⁻⁷ M and 5×10⁻⁸ M, 5×10⁻⁷ M and 1×10⁻⁸ M, between 5×10⁻⁷ M and 5×10⁻⁹ M, between 5×10⁻⁷ M and 1×10⁻⁹ M, between 5×10⁻⁷ M and 5×10⁻¹⁰ M, between 5×10⁻⁷ M and 1×10⁻¹⁰ M, between 5×10⁻⁷ M and 5×10⁻¹¹ M, between 5×10⁻⁷ M and 1×10⁻¹¹ M, between 5×10⁻⁷ M and 5×10⁻¹² M, between 5×10⁻⁷ M and 1×10⁻¹² M, between 1×10⁻⁷ M and 5×10⁻⁸ M, 1×10⁻⁷ M and 1×10⁻⁸ M, between 1×10⁻⁷ M and 5×10⁻⁹ M, between 1×10⁻⁷ M and 1×10⁻⁹ M, between 1×10⁻⁷ M and 5×10⁻¹⁰ M, between 1×10⁻⁷ M and 1×10⁻¹⁰ M, between 1×10⁻⁷ M and 5×10⁻¹¹ M, between 1×10⁻⁷ M and 1×10⁻¹¹ M, between 1×10⁻⁷ M and 5×10⁻¹² M, between 1×10⁻⁷ M and 1×10⁻¹² M, between 5×10⁻⁸ M and 1×10⁻⁸ M, between 5×10⁻⁸ M and 5×10⁻⁹ M, between 5×10⁻⁸ M and 1×10⁻⁹ M, between 5×10⁻⁸ M and 5×10⁻¹⁰ M, between 5×10⁻⁸ M and 1×10⁻¹⁰ M, between 5×10⁻⁸ M and 5×10⁻¹¹ M, between 5×10⁻⁸ M and 1×10⁻¹¹ M, between 5×10⁻⁸ M and 5×10⁻¹² M, between 5×10⁻⁸ M and 1×10⁻¹² M, 1×10⁻⁸ M and 5×10⁻⁹ M, between 1×10⁻⁸ M and 1×10⁻⁹ M, between 1×10⁻⁸ M and 5×10⁻¹⁰ M, between 1×10⁻⁸ M and 1×10⁻¹⁰ M, between 1×10⁻⁸ M and 5×10⁻¹¹ M, between 1×10⁻⁸ M and 1×10⁻¹¹ M, between 1×10⁻⁸ M and 5×10⁻¹² M, between 1×10⁻⁸ M and 1×10⁻¹² M, between 5×10⁻⁹ M and 1×10⁻⁹ M, between 5×10⁻⁹ M and 5×10⁻¹⁰ M, between 5×10⁻⁹ M and 1×10⁻¹⁰ M, between 5×10⁻⁹ M and 5×10⁻¹¹ M, between 5×10⁻⁹ M and 1×10⁻¹¹ M, between 5×10⁻⁹ M and 5×10⁻¹² M, between 5×10⁻⁹ M and 1×10⁻¹² M, between 1×10⁻⁹ M and 5×10⁻¹⁰ M, between 1×10⁻⁹ M and 1×10⁻¹⁰ M, between 1×10⁻⁹ M and 5×10⁻¹¹ M, between 1×10⁻⁹ M and 1×10⁻¹¹ M, between 1×10⁻⁹ M and 5×10⁻¹² M, between 1×10⁻⁹ M and 1×10⁻¹² M, between 5×10⁻¹⁰ M and 1×10⁻¹⁰ M, between 5×10⁻¹⁰ M and 5×10⁻¹¹ M, between, 1×10⁻¹⁰ M and 5×10⁻¹¹ M, 1×10⁻¹⁰ M and 1×10⁻¹¹ M, between 1×10⁻¹⁰ M and 5×10⁻¹² M, between 1×10⁻¹⁰ M and 1×10⁻¹² M, between 5×10⁻¹¹ M and 1×10⁻¹² M, between 5×10⁻¹¹ M and 5×10⁻¹² M, between 5×10⁻¹¹ M and 1×10⁻¹² M, between 1×10⁻¹¹ M and 5×10⁻¹² M, or between 1×10⁻¹¹ M and 1×10⁻¹² M, as measured by biolayer interferometry. In some embodiments, an IL13R/IL4R contiguous polypeptide binds to canine IL13 and/or IL4, feline IL13 and/or IL4, and/or equine IL13 and/or IL4.

To “reduce” or “inhibit” means to decrease, reduce, or arrest an activity, function, or amount as compared to a reference. In some embodiments, by “reduce” or “inhibit” is meant the ability to cause an overall decrease of 20% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control dose (such as a placebo) over the same period of time. A “reference” as used herein, refers to any sample, standard, or level that is used for comparison purposes. A reference may be obtained from a healthy or non-diseased sample. In some examples, a reference is obtained from a non-diseased or non-treated sample of a companion animal. In some examples, a reference is obtained from one or more healthy animals of a particular species, which are not the animal being tested or treated.

The term “substantially reduced,” as used herein, denotes a sufficiently high degree of reduction between a numeric value and a reference numeric value such that one of skill in the art would consider the difference between the two values to be of statistical significance within the context of the biological characteristic measured by said values. In some embodiments, the substantially reduced numeric values is reduced by greater than about any one of 10%, 15% 20%, 25%, 30%, 35%, 40%, 45%, 50%, 60%, 70%, 80%, 90%, or 100% compared to the reference value.

In some embodiments, an IL13R/IL4R contiguous polypeptide may reduce IL13 and/or IL4 signaling in a companion animal species by at least 10%, at least 15%, at least 20%, at least 25%, at least 30%, at least 35%, at least 40%, at least 45%, at least 50%, at least 60%, at least 70%, at least 80%, at least 90%, or 100% compared to IL13 and/or IL4 signaling in the absence of the fusion molecule. In some embodiments, signaling is measured by a reduction in IL4-dependent TF-1 cell proliferation. In some embodiments, the reduction in IL13 and/or IL4 signaling or the reduction in proliferation is between 10% and 15%, between 10% and 20%, between 10% and 25%, between 10% and 30%, between 10% and 35%, between 10% and 40%, between 10% and 45%, between 10% and 50%, between 10% and 60%, between 10% and 70%, between 10% and 80%, between 10% and 90%, between 10% and 100%, between 15% and 20%, between 15% and 25%, between 15% and 30%, between 15% and 35%, between 15% and 40%, between 15% and 45%, between 15% and 50%, between 15% and 60%, between 15% and 70%, between 15% and 80%, between 15% and 90%, between 15% and 100%, between 20% and 25%, between 20% and 30%, between 20% and 35%, between 20% and 40%, between 20% and 45%, between 20% and 50%, between 20% and 60%, between 20% and 70%, between 20% and 80%, between 20% and 90%, between 20% and 100%, between 25% and 30%, between 25% and 35%, between 25% and 40%, between 25% and 45%, between 25% and 50%, between 25% and 60%, between 25% and 70%, between 25% and 80%, between 25% and 90%, between 25% and 100%, between 30% and 35%, between 30% and 40%, between 30% and 45%, between 30% and 50%, between 30% and 60%, between 30% and 70%, between 30% and 80%, between 30% and 90%, between 30% and 100%, between 35% and 40%, between 35% and 45%, between 35% and 50%, between 35% and 60%, between 35% and 70%, between 35% and 80%, between 35% and 90%, between 35% and 100%, between 40% and 45%, between 40% and 50%, between 40% and 60%, between 40% and 70%, between 40% and 80%, between 40% and 90%, between 40% and 100%, between 45% and 50%, between 45% and 60%, between 45% and 70%, between 45% and 80%, between 45% and 90%, between 45% and 100%, between 50% and 60%, between 50% and 70%, between 50% and 80%, between 50% and 90%, between 50% and 100%, between 60% and 70%, between 60% and 80%, between 60% and 90%, between 60% and 100%, between 70% and 80%, between 70% and 90%, between 70% and 100%, between 80% and 90%, between 80% and 100%, or between 90% and 100%.

### Pharmaceutical Compositions

The terms “pharmaceutical formulation” and “pharmaceutical composition” refer to a preparation which is in such form as to permit the biological activity of the active ingredient(s) to be effective, and which contains no additional components that are unacceptably toxic to a subject to which the formulation would be administered.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. Examples of pharmaceutically acceptable carriers include alumina; aluminum stearate; lecithin; serum proteins, such as human serum albumin, canine or other animal albumin; buffers such as phosphate, citrate, tromethamine or HEPES buffers; glycine; sorbic acid; potassium sorbate; partial glyceride mixtures of saturated vegetable fatty acids; water; salts or electrolytes, such as protamine sulfate, disodium hydrogen phosphate, potassium hydrogen phosphate, sodium chloride, zinc salts, colloidal silica, or magnesium trisilicate; polyvinyl pyrrolidone, cellulose-based substances; polyethylene glycol; sucrose; mannitol; or amino acids including, but not limited to, arginine.

The pharmaceutical composition can be stored in lyophilized form. Thus, in some embodiments, the preparation process includes a lyophilization step. The lyophilized composition may then be reformulated, typically as an aqueous composition suitable for parenteral administration, prior to administration to the dog, cat, or horse. In other embodiments, particularly where the fusion molecule is highly stable to thermal and oxidative denaturation, the pharmaceutical composition can be stored as a liquid, i.e., as an aqueous composition, which may be administered directly, or with appropriate dilution, to the dog, cat, or horse. A lyophilized composition can be reconstituted with sterile Water for Injection (WFI). Bacteriostatic reagents, such benzyl alcohol, may be included. Thus, the invention provides pharmaceutical compositions in solid or liquid form.

The pH of the pharmaceutical compositions may be in the range of from about pH 5 to about pH 8, when administered. The compositions of the invention are sterile if they are to be used for therapeutic purposes. Sterility can be achieved by any of several means known in the art, including by filtration through sterile filtration membranes (e.g., 0.2 micron membranes). Sterility may be maintained with or without anti-bacterial agents.

### Uses of IL13R/IL4R Contiguous Polypeptides and Pharmaceutical Compositions

The IL13R/IL4R contiguous polypeptides or pharmaceutical compositions comprising the IL13R/IL4R contiguous polypeptides of the invention may be useful for treating an IL13- and/or IL4-induced condition. As used herein, an “IL13 or IL4-induced condition” means a disease associated with, caused by, or characterized by, elevated levels or altered distribution of IL13 or IL4. Such IL13 and/or IL4-induced conditions include, but are not limited to, a pruritic or an allergic disease. In some embodiments, the IL13- and/or IL4-induced condition is atopic dermatitis, pruritus, asthma, psoriasis, scleroderma, or eczema. An IL13- or IL4-induced condition may be exhibited in a companion animal, including, but not limited to, canine, feline, or equine.

As used herein, “treatment” is an approach for obtaining beneficial or desired clinical results. “Treatment” as used herein, covers any administration or application of a therapeutic for disease in a mammal, including a companion animal. For purposes of this disclosure, beneficial or desired clinical results include, but are not limited to, any one or more of: alleviation of one or more symptoms, diminishment of extent of disease, preventing or delaying spread of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

In some embodiments, an IL13R/IL4R contiguous polypeptide or pharmaceutical compositions comprising it can be utilized in accordance with the methods herein to treat IL13- or IL4-induced conditions. In some embodiments, an IL13R/IL4R contiguous polypeptide or pharmaceutical compositions is administered to a companion animal, such as a canine, a feline, or equine, to treat an IL13- and IL4-induced condition.

A “therapeutically effective amount” of a substance/molecule, agonist or antagonist may vary according to factors such as the type of disease to be treated, the disease state, the severity and course of the disease, the type of therapeutic purpose, any previous therapy, the clinical history, the response to prior treatment, the discretion of the attending veterinarian, age, sex, and weight of the animal, and the ability of the substance/molecule, agonist or antagonist to elicit a desired response in the animal. A therapeutically effective amount is also one in which any toxic or detrimental effects of the substance/molecule, agonist or antagonist are outweighed by the therapeutically beneficial effects. A therapeutically effective amount may be delivered in one or more administrations. A therapeutically effective amount refers to an amount effective, at dosages and for periods of time necessary, to achieve the desired therapeutic or prophylactic result.

In some embodiments, IL13R/IL4R contiguous polypeptide or pharmaceutical composition comprising an IL13R/IL4R contiguous polypeptide is administered parenterally, by subcutaneous administration, intravenous infusion, or intramuscular injection. In some embodiments, an IL13R/IL4R contiguous polypeptide or pharmaceutical composition comprising an IL13R/IL4R contiguous polypeptide is administered as a bolus injection or by continuous infusion over a period of time. In some embodiments, an IL13R/IL4R contiguous polypeptide or pharmaceutical composition comprising an IL13R/IL4R contiguous polypeptide is administered by an intramuscular, an intraperitoneal, an intracerebrospinal, a subcutaneous, an intra-arterial, an intrasynovial, an intrathecal, or an inhalation route.

An IL13R/IL4R contiguous polypeptide described herein may be administered in an amount in the range of 0.1 mg/kg body weight to 100 mg/kg body weight per dose. In some embodiments, anti-IL13/IL4 fusion may be administered in an amount in the range of 0.5 mg/kg body weight to 50 mg/kg body weight per dose. In some embodiments, anti-IL13/IL4 fusion may be administered in an amount in the range of 1 mg/kg body weight to 10 mg/kg body weight per dose. In some embodiments, fusion molecule may be administered in an amount in the range of 0.5 mg/kg body weight to 100 mg/kg body, in the range of 1 mg/kg body weight to 100 mg/kg body weight, in the range of 5 mg/kg body weight to 100 mg/kg body weight, in the range of 10 mg/kg body weight to 100 mg/kg body weight, in the range of 20 mg/kg body weight to 100 mg/kg body weight, in the range of 50 mg/kg body weight to 100 mg/kg body weight, in the range of 1 mg/kg body weight to 10 mg/kg body weight, in the range of 5 mg/kg body weight to 10 mg/kg body weight, in the range of 0.5 mg/kg body weight to 10 mg/kg body weight, or in the range of 5 mg/kg body weight to 50 mg/kg body weight.

An IL13R/IL4R contiguous polypeptide or a pharmaceutical composition comprising an IL13R/IL4R contiguous polypeptide can be administered to a companion animal at one time or over a series of treatments. For example, IL13R/IL4R contiguous polypeptide or a pharmaceutical composition comprising an IL13R and IL4R may be administered at least once, more than once, at least twice, at least three times, at least four times, or at least five times.

In some embodiments, the dose is administered once per week for at least two or three consecutive weeks, and in some embodiments, this cycle of treatment is repeated two or more times, optionally interspersed with one or more weeks of no treatment. In other embodiments, the therapeutically effective dose is administered once per day for two to five consecutive days, and in some embodiments, this cycle of treatment is repeated two or more times, optionally interspersed with one or more days or weeks of no treatment.

Administration “in combination with” one or more further therapeutic agents includes simultaneous (concurrent) and consecutive or sequential administration in any order. The term “concurrently” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered with a time separation of no more than about a specified number of minutes. The term “sequentially” is used herein to refer to administration of two or more therapeutic agents where the administration of one or more agent(s) continues after discontinuing the administration of one or more other agent(s), or wherein administration of one or more agent(s) begins before the administration of one or more other agent(s). For example, administration of the two or more therapeutic agents are administered with a time separation of more than about a specified number of minutes. As used herein, “in conjunction with” refers to administration of one treatment modality in addition to another treatment modality. As such, “in conjunction with” refers to administration of one treatment modality before, during or after administration of the other treatment modality to the animal.

In some embodiments, the method comprises administering in combination with an IL13R/IL4R contiguous polypeptide or a pharmaceutical composition comprising an IL13R/IL4R contiguous polypeptide, a Jak inhibitor, a PI3K inhibitor, an AKT inhibitor, or a MAPK inhibitor. In some embodiments, the method comprises administering in combination with an IL13R/IL4R contiguous polypeptide or a pharmaceutical composition comprising an IL13R/IL4R contiguous polypeptide, an anti-IL17 antibody, an anti-TNFα antibody, an anti-CD20 antibody, an anti-CD19 antibody, an anti-CD25 antibody, an anti-IL31 antibody, an anti-IL23 antibody, an anti-IgE antibody, an anti-CD11a antibody, anti-IL6R antibody, anti-α4-Integrin antibody, an anti-IL12 antibody, an anti-IL1β antibody, or an anti-BlyS antibody.

Provided herein are methods of exposing to a cell an IL13R/IL4R contiguous polypeptide or a pharmaceutical composition comprising an IL13R/IL4R contiguous polypeptide under conditions permissive for binding to IL13 or IL4. In some embodiments, the cell is exposed to the IL13R/IL4R contiguous polypeptide or pharmaceutical composition ex vivo. In some embodiments, the cell is exposed to the IL13R/IL4R contiguous polypeptide or pharmaceutical composition in vivo. In some embodiments, a cell is exposed to the IL13R/IL4R contiguous polypeptide. In some embodiments, a cell is exposed to the IL13R/IL4R contiguous polypeptide or the pharmaceutical composition under conditions permissive for binding of the fusion molecule to extracellular IL13 or IL4. In some embodiments, a cell may be exposed in vivo to the IL13R/IL4R contiguous polypeptide or the pharmaceutical composition by any one or more of the administration methods described herein, including but not limited to, intraperitoneal, intramuscular, intravenous injection into the subject. In some embodiments, a cell may be exposed ex vivo to the IL13R/IL4R contiguous polypeptide or the pharmaceutical composition by exposing the cell to a culture medium comprising the fusion molecule or the pharmaceutical composition. In some embodiments, the permeability of the cell membrane may be affected using any number of methods understood by those of skill in the art (such as electroporating the cells or exposing the cells to a solution containing calcium chloride) before exposing the cell to a culture medium comprising the fusion molecule or the pharmaceutical composition.

In some embodiments, the exposure results in a reduction of IL13 or IL4 signaling function by the cell. In some embodiments, an IL13R/IL4R contiguous polypeptide may reduce IL13 or IL4 signaling in a cell by at least 10%, at least 15%, at least 20%, at least 25%, at least 30%, at least 35%, at least 40%, at least 45%, at least 50%, at least 60%, at least 70%, at least 80%, at least 90%, or 100% compared to IL13 or IL4 signaling function in the absence of the IL13R/IL4R contiguous polypeptide. In some embodiments, the reduction in IL13 or IL4 signaling or the reduction in TF-1 proliferation is between 10% and 15%, between 10% and 20%, between 10% and 25%, between 10% and 30%, between 10% and 35%, between 10% and 40%, between 10% and 45%, between 10% and 50%, between 10% and 60%, between 10% and 70%, between 10% and 80%, between 10% and 90%, between 10% and 100%, between 15% and 20%, between 15% and 25%, between 15% and 30%, between 15% and 35%, between 15% and 40%, between 15% and 45%, between 15% and 50%, between 15% and 60%, between 15% and 70%, between 15% and 80%, between 15% and 90%, between 15% and 100%, between 20% and 25%, between 20% and 30%, between 20% and 35%, between 20% and 40%, between 20% and 45%, between 20% and 50%, between 20% and 60%, between 20% and 70%, between 20% and 80%, between 20% and 90%, between 20% and 100%, between 25% and 30%, between 25% and 35%, between 25% and 40%, between 25% and 45%, between 25% and 50%, between 25% and 60%, between 25% and 70%, between 25% and 80%, between 25% and 90%, between 25% and 100%, between 30% and 35%, between 30% and 40%, between 30% and 45%, between 30% and 50%, between 30% and 60%, between 30% and 70%, between 30% and 80%, between 30% and 90%, between 30% and 100%, between 35% and 40%, between 35% and 45%, between 35% and 50%, between 35% and 60%, between 35% and 70%, between 35% and 80%, between 35% and 90%, between 35% and 100%, between 40% and 45%, between 40% and 50%, between 40% and 60%, between 40% and 70%, between 40% and 80%, between 40% and 90%, between 40% and 100%, between 45% and 50%, between 45% and 60%, between 45% and 70%, between 45% and 80%, between 45% and 90%, between 45% and 100%, between 50% and 60%, between 50% and 70%, between 50% and 80%, between 50% and 90%, between 50% and 100%, between 60% and 70%, between 60% and 80%, between 60% and 90%, between 60% and 100%, between 70% and 80%, between 70% and 90%, between 70% and 100%, between 80% and 90%, between 80% and 100%, or between 90% and 100%.

Provided herein are methods of using the IL13R/IL4R contiguous polypeptide, polypeptides and polynucleotides for detection, diagnosis and monitoring of an IL13- or IL4-induced condition. Provided herein are methods of determining whether a companion animal will respond to IL13R/IL4R contiguous polypeptide therapy. In some embodiments, the method comprises detecting whether the animal has cells that express IL13 or IL4 using an IL13R/IL4R contiguous polypeptide. In some embodiments, the method of detection comprises contacting the sample with an antibody, polypeptide, or polynucleotide and determining whether the level of binding differs from that of a reference or comparison sample (such as a control). In some embodiments, the method may be useful to determine whether the IL13R/IL4R contiguous polypeptides described herein are an appropriate treatment for the subject animal.

In some embodiments, the sample is a biological sample. The term “biological sample” means a quantity of a substance from a living thing or formerly living thing. In some embodiments, the biological sample is a cell or cell/tissue lysate. In some embodiments, the biological sample includes, but is not limited to, blood, (for example, whole blood), plasma, serum, urine, synovial fluid, and epithelial cells.

In some embodiments, the cells or cell/tissue lysate are contacted with an IL13R/IL4R contiguous polypeptide and the binding between the IL13R/IL4R contiguous polypeptide and the cell is determined. When the test cells show binding activity as compared to a reference cell of the same tissue type, it may indicate that the subject would benefit from treatment with an IL13R/IL4R contiguous polypeptide. In some embodiments, the test cells are from tissue of a companion animal.

Various methods known in the art for detecting specific antibody-antigen binding can be used. Exemplary immunoassays which can be conducted include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, can be attached to the subject antibodies and is selected to meet the needs of various uses of the method which are often dictated by the availability of assay equipment and compatible immunoassay procedures. Appropriate labels include, without limitation, radionuclides (for example ¹²⁵I, ¹³¹I, ³⁵S, ³H, or ³²P), enzymes (for example, alkaline phosphatase, horseradish peroxidase, luciferase, or p-galactosidase), fluorescent moieties or proteins (for example, fluorescein, rhodamine, phycoerythrin, GFP, or BFP), or luminescent moieties (for example, Qdot™ nanoparticles supplied by the Quantum Dot Corporation, Palo Alto, Calif.). General techniques to be used in performing the various immunoassays noted above are known to those of ordinary skill in the art.

For purposes of diagnosis, the IL13R/IL4R contiguous polypeptide can be labeled with a detectable moiety including but not limited to radioisotopes, fluorescent labels, and various enzyme-substrate labels know in the art. Methods of conjugating labels to polypeptides are known in the art. In some embodiments, the IL13R/IL4R contiguous polypeptide need not be labeled, and the presence thereof can be detected, for example, using an antibody that binds to the IL13R/IL4R contiguous polypeptide. In some embodiments, the IL13R/IL4R contiguous polypeptide can be employed in any known assay method, such as competitive binding assays, direct and indirect sandwich assays, and immunoprecipitation assays. Zola, Monoclonal Antibodies: A Manual of Techniques, pp. 147-158 (CRC Press, Inc. 1987). The anti-IL13 and IL4 antibodies and polypeptides can also be used for in vivo diagnostic assays, such as in vivo imaging. Generally, the antibody or the polypeptide is labeled with a radionuclide (such as ¹¹¹In, ⁹⁹Tc, ¹⁴C, ¹³¹I, ¹²⁵I, ³H, or any other radionuclide label, including those outlined herein) so that the cells or tissue of interest can be localized using immunoscintiography. The IL13R/IL4R contiguous polypeptide may also be used as staining reagent in pathology using techniques well known in the art.

In some embodiments, a IL13R/IL4R contiguous polypeptide is used for a diagnostic and a IL13R/IL4R contiguous polypeptide is used as a therapeutic. In some embodiments, the first and second IL13R/IL4R contiguous polypeptide are different.

The following examples illustrate particular aspects of the disclosure and are not intended in any way to limit the disclosure.

### EXAMPLES

### Example 1

### Expression and Purification of Canine IL4 and IL13

A nucleotide sequence encoding canine IL13 protein (SEQ ID NO: 4) was synthesized with poly-His tag on the C-terminal end and cloned into a mammalian expression vector and transfected to 293 cells or CHOS. The same method was used to clone and express a nucleotide sequence encoding canine IL4 protein (SEQ ID NO: 1) with a poly-His tag on the C-terminal end.

The supernatant containing canine IL13 protein was collected and filtered. Canine IL13 was affinity purified using Ni-NTA column (CaptivA® Protein A Affinity Resin, Repligen). The same method was used to purify canine IL4.

### Example 2

### Extracellular Domains of IL13R and IL4R

Extracellular domains of canine, feline, and equine IL4R that are responsible for binding canine, feline and equine IL4 and/or IL13 were identified and boundaries were defined. Full-length extracellular domains of canine IL4R, feline IL4R, and equine IL4 were identified as SEQ ID NO: 23, SEQ ID NO: 25, and SEQ ID NO: 27, respectively. Extracellular domain fragments of canine IL4R, feline IL4R, and equine IL4R postulated to retain biological activity were identified as SEQ ID NO: 33, SEQ ID NO: 35, and SEQ ID NO: 37, respectively.

Extracellular domains of canine, feline, and equine IL13R that are responsible for binding canine, feline, and equine IL4 and/or IL13 were identified and boundaries were defined. Full length extracellular domains of canine IL13R, feline IL13R, and equine IL13R were identified as SEQ ID NO: 22, SEQ ID NO: 24, and SEQ ID NO: 26, respectively. Extracellular domain fragments of canine IL13R, feline IL13R, and equine IL13R postulated to retain biological activity were identified as SEQ ID NO: 32, SEQ ID NO: 34, and SEQ ID NO: 36, respectively.

An unpaired cysteine (Cys) in canine IL13R (at position 18 of SEQ ID NO: 22), feline IL13R (at position 18 of SEQ ID NO: 24), and equine IL13R (at position 18 of SEQ ID NO: 26) was identified informatically and determined as embedded (unexposed) based on 3-D modeling. It is unlikely that the unpaired cysteine will form disulfide bonds and the likelihood of aggregation is low. Thus, site-directed mutagenesis of this Cys residue was not introduced.

### Example 3

Expression and Purification of Canine IL13R/IL4R Contiguous Polypeptides from CHO Cells

Nucleotide sequences encoding canine IL13R/IL4R contiguous polypeptides linked to Fc IgGB were designed with a signal sequence. For contiguous polypeptide “IL13R-IL4R-IgGB” (SEQ ID NO: 20), the extracellular domain of IL13R (SEQ ID NO: 22) precedes the extracellular domain of IL4R (SEQ ID NO: 23). For contiguous polypeptide IL4R-IL13R-IgGB (SEQ ID NO: 21), the extracellular domain of IL4R precedes the extracellular domain of IL13R.

The nucleotide sequences were synthesized chemically and inserted into an expression vector suitable for transfection into a CHO host cell. After transfection into CHO cells, the fusion proteins were secreted from the cell. For example, fusion protein was purified by single step Protein A column chromatography.

Each of IL13R-IL4R-IgGB and IL4R-IL13R-IgGB may be expressed and purified in a single step with a protein A column or other chromatographic methods, such as ion exchange column chromatography, hydrophobic interaction column chromatography, mixed mode column chromatography such as CHT, or multimodal mode column chromatography such as CaptoMMC. Low pH or other viral inactivation and viral removal steps can be applied. The purified protein may be admixed with excipients, and sterilized by filtration to prepare a pharmaceutical composition of the invention. The pharmaceutical composition may be administered to a dog with an atopic dermatitis or asthma in an amount sufficient to bind and/or inhibit either IL13 and/or IL4.

The vectors were then used to perform pilot-scale transfection in CHO-S cells using the FreestyleMax™ transfection reagent (Life Technologies). The supernatant was harvested by clarifying the conditioned media. Protein was purified with a single pass Protein A chromatography step and used for further investigation.

### Example 4

### Demonstration of IL13 and IL4 Binding Activity

This example demonstrates that both IL13R-IL4R-IgGB (SEQ ID NO:20) and IL4R-IL13R-IgGB (SEQ ID NO:21) bind canine IL4 and IL13 with kinetics requisite for therapeutic activity.

The binding analysis was performed using a biosensor Octet as follows. Briefly, canine IL4 (produced using 293 cells) was biotinylated. The free unreacted biotin was removed from biotinylated IL4 by extensive dialysis. Biotinylated canine IL4 was captured on streptavidin sensor tips. The IL4 association with various concentrations (12, 16, and 44 nM) of IL13R-IL4R-IgGB (SEQ ID NO:20) was monitored for ninety seconds. Dissociation was monitored for 600 seconds. A buffer only blank curve was subtracted to correct for any drift. The data were fit to a 1:1 binding model using ForteBio™ data analysis software to determine the kₒₙ, k_{off}, and the Kd. The buffer for dilutions and all binding steps was: 20 mM phosphate, 150 mM NaCl, pH 7.2. The Kd for IL13R-IL4R-IgGB and ligand IL4 was 8×10⁻¹¹.

The canine IL4 association with various concentrations (40.7, and 140 nM) of IL4R-IL13R-IgGB (SEQ ID NO:21) was monitored for ninety seconds. Dissociation was monitored for 600 seconds. A buffer only blank curve was subtracted to correct for any drift. The data were fit to a 1:1 binding model using ForteBio™ data analysis software to determine the kon, koff, and the Kd. The buffer for dilutions and all binding steps was: 20 mM phosphate, 150 mM NaCl, pH 7.2. The Kd for IL4R-IL13R-IgGB and ligand IL4 was 1.1×10⁻¹¹.

Canine IL4 and canine IL13 with C-terminal polyHis tag was expressed and purified from 293 cells. EZ-Link NHS-LC-biotin was obtained from Thermo Scientific (Cat. #21336), and Streptavidin biosensors was obtained from ForteBio (Cat. #18-509).

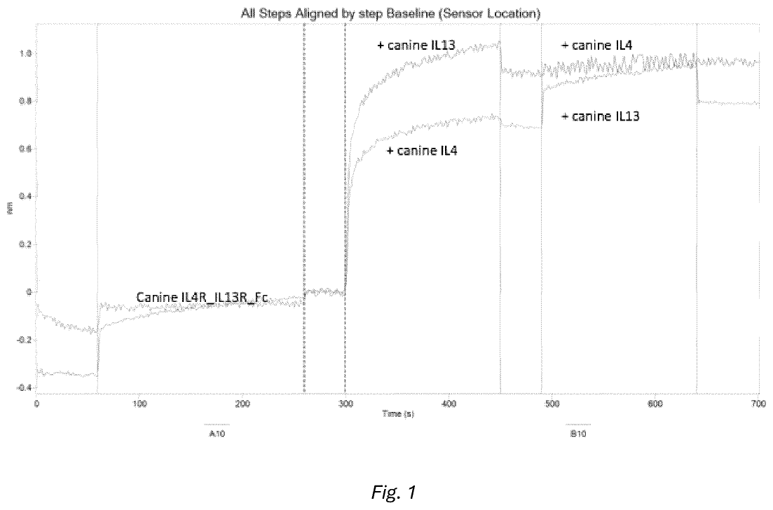

IL4 and IL13 sequential binding experiments with IL13R-IL4R-IgGB (SEQ ID NO:20) were performed. Biotinylated canine IL13R-IL4R-IgGB was captured on streptavidin sensor tips. Canine IL13R-IL4R-IgGB was exposed to either (1) canine IL4 followed by IL13 or (2) canine IL13 followed by IL4 using concentrations of 30 μg/mL of IL4 and IL13 in PBS (FIG. 1). The experiments demonstrated that once IL13R-IL4R-IgGB bound to IL13, it may not bind to IL4, and that once bound to IL4, its ability to bind IL13 is reduced.

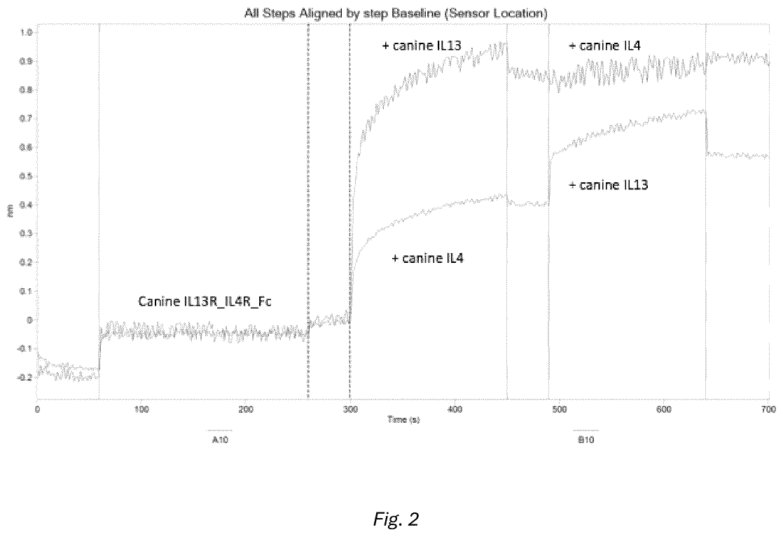

IL4 and IL13 sequential binding experiments with IL4R-IL13R-IgGB (SEQ ID NO:21) were performed. Biotinylated canine IL4R-IL13R-IgGB was captured on streptavidin sensor tips. Canine IL4R-IL13R-IgGB was exposed to either (1) canine IL4 followed by IL13 or (2) canine IL13 followed by IL4 using concentrations of 30 μg/mL of IL4 and IL13 in PBS (FIG. 2). These experiments demonstrated that once IL4R-IL13R-IgGB bound to IL13, it may not bind to IL4, and that once bound to IL4, its ability to bind IL13 is reduced.

The tight binding of IL13R-IL4R-IgGB and IL4R-IL13R-IgGB to IL4 or IL13 is thought to be due to simultaneous binding contributions made by both IL4R and IL13R.

### Example 5

### Cellular Functional Activity of Canine IL4R-IL13R-Fc (SINK)

TF1 cells (ATCC cat #CRL-2003), a human Erythroleukemic cell line which expresses endogenous interleukin 4 receptors on cell surface, was used in a proliferation assay. Cells grown in RPMI1640 (Gibco, Cat #11875) supplemented with 10% Fetal Bovine Serum, heat inactivated (Sigma, Cat #2868) and 2 nM/ml Human GM-CSF (R&D System, Cat #215-GM-010) at exponential growth phase were used for the assay. Cells were washed with PBS twice and resuspended in above medium without GM-CSF. 20,000 cells per well were plated in a 96-well plate (Corning, Cat #3610). Canine IL4R-IL13R-Fc (SINK) was added at a series of dilutions followed by addition of canine IL4 (Sino Biological Inc, Cat #70021-DNAE-5) at 50 ng/ml. The cells were incubated in 37° C., 5% CO2 for 48 hours in a total volume of 100 μl. At the end of the incubation, the cells were cooled in room temperature and assayed for proliferation/variability by measuring cellular ATP content using CellTiter-Glo® Luminescent Cell Viability Assay (Promega, Cat #G7570).

In this assay, 100 μl premixed reagent A and B were added to each well. After shaking on an orbital shaker for 2 mins, the cells were lysed. Mono-oxygenation of luciferin was catalyzed by luciferase in the presence of Mg2+ and ATP that presented in cells, resulting in the generation of a luminescent signal proportional to the amount of ATP in the cells. The amount of ATP is directly proportional to the number of cells present in culture. The plate was incubated at room temperature for 10 minutes to stabilize the luminescent signal and luminescence was detected using a Synergy HT microplate reader (Biotek, Winooski, Vt.).

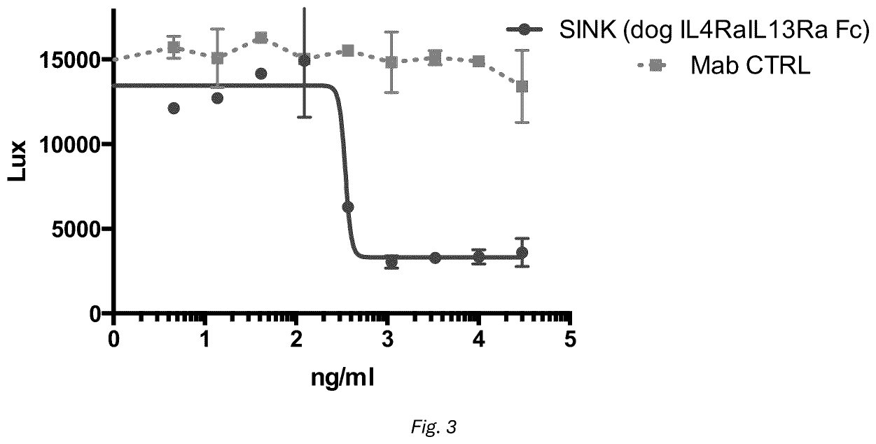

The data were analyzed using 4 parameter logistic fit and IC50 is 2.0 nM. See FIG. 3.

What is claimed is:

1. A method of treating a companion animal species having an IL13-induced condition and/or an IL4-induced condition, the method comprising administering to the companion animal species a therapeutically effective amount of a polypeptide comprising an amino acid sequence at least 95% identical to a sequence selected from the group consisting of SEQ ID NO: 13, SEQ ID NO: 14, SEQ ID NO: 15, SEQ ID NO: 16, SEQ ID NO: 17, SEQ ID NO 18, SEQ ID NO: 19, SEQ ID NO: 20, SEQ ID NO: 21, SEQ ID NO: 28, SEQ ID NO: 29, SEQ ID NO: 30, and SEQ ID NO: 31.

2. The method of claim 1, wherein the companion animal species is canine, feline, or equine.

3. The method of claim 1, wherein the IL13-induced condition and/or the IL4-induced condition is a pruritic or allergic condition.

4. The method of claim 3, wherein the pruritic or allergic condition is selected from the group consisting of atopic dermatitis, pruritus, asthma, psoriasis, scleroderma, and eczema.

5. The method of claim 1, wherein the method further comprises administering:
a) one or more inhibitors selected from a Jak inhibitor, a PI3K inhibitor, and AKT inhibitor, and a MAPK inhibitor; and/or
b) one or more antibodies selected from an anti-IL17 antibody, an anti-IL31 antibody, an anti-TNFα antibody, an anti-CD20 antibody, an anti-CD19 antibody, and anti-CD25 antibody, an anti-IL4 antibody, an anti-IL13 antibody, an anti-IL23 antibody, an anti-IgE antibody, an anti-CD11a antibody, an anti-IL6R antibody, an anti-α4-Integrin antibody, an anti-IL12 antibody, an anti-IL1β antibody, and an anti-BlyS antibody.

6. The method of claim 1, wherein the polypeptide comprises a post-expression modification.

7. The method of claim 6, wherein the modification comprises glycosylation, sialyation, acetylation, and/or phosphorylation.

8. The method of claim 1, wherein the polypeptide comprises an amino acid sequence selected from SEQ ID NO: 13, SEQ ID NO: 14, SEQ ID NO: 15, SEQ ID NO: 16, and SEQ ID NO: 17.

9. The method of claim 8, wherein the polypeptide binds to IL13 of a companion animal species with a dissociation constant (Kd) of less than 5×10-6 M, as measured by biolayer interferometry.

10. The method of claim 8, wherein the polypeptide binds to IL4 of a companion animal species with a dissociation constant (Kd) of less than 5×10-6 M, as measured by biolayer interferometry.

11. The method of claim 1, wherein the polypeptide comprises SEQ ID NO: 15.

12. The method of claim 1, wherein the polypeptide comprises an amino acid sequence at least 98% identical to a sequence selected from the group consisting of SEQ ID NO: 13, SEQ ID NO: 14, SEQ ID NO: 15, SEQ ID NO: 16, SEQ ID NO: 17, SEQ ID NO 18, SEQ ID NO: 19, SEQ ID NO: 20, SEQ ID NO: 21, SEQ ID NO: 28, SEQ ID NO: 29, SEQ ID NO: 30, and SEQ ID NO: 31.

13. The method of claim 1, wherein the polypeptide comprises an amino acid sequence at least 99% identical to a sequence selected from the group consisting of SEQ ID NO: 13, SEQ ID NO: 14, SEQ ID NO: 15, SEQ ID NO: 16, SEQ ID NO: 17, SEQ ID NO 18, SEQ ID NO: 19, SEQ ID NO: 20, SEQ ID NO: 21, SEQ ID NO: 28, SEQ ID NO: 29, SEQ ID NO: 30, and SEQ ID NO: 31.

14. The method of claim 1, wherein the polypeptide optionally comprises an amino acid substitution.

15. The method of claim 14, wherein the amino acid substitution is a conservative amino acid substitution.
